(19) United States
(12) Patent Application Publication
(10) Pub. No.: US 2010/0173827 A1
(43) Pub. Date: Jul. 8, 2010

(54) ROLE OF PROLINE RICH PEPTIDES IN CELLULAR COMMUNICATION MECHANISMS AND TREATMENT OF DISEASES

(76) Inventors: Jerzy Alexander Georgiades, Houston, TX (US); Izolda Maria Schroeder-Georgiades, Houston, TX (US)

Correspondence Address: CHALKER FLORES, LLP, 2711 LBJ FRWY, Suite 1036, DALLAS, TX, 75234, US

(21) Appl. No.: 12/639,885

(22) Filed: Dec. 16, 2009

Related U.S. Application Data
(60) Provisional application No. 61/201,909, filed on Dec. 16, 2008.

(51) Int. Cl. A61K 38/40; G01N 33/53; A61K 38/02; A61K 38/10; A61K 38/08; A61K 38/07; A61P 9/00; A61P 25/16; A61P 31/18; A61P 25/00; A61P 25/28

(52) U.S. Cl. 514/6; 435/7.92; 514/2; 514/14; 514/15; 514/17; 514/18

(57) ABSTRACT

The present invention includes peptide compositions for improving cellular communication mechanisms, repairing metabolic errors, and for treatment of AD, dementia and other neurological conditions. The proline rich peptide complexes (PRPC) of the present invention are isolated from colostrinin and can be added in nutritional and other food supplements. Additionally, the present invention describes novel communication system governing circa two hundreds of different types of cells in each mammal that modulates the activities of the organism through the biological computer present in every living cell. Different type of cells can communicate with other through biological computer connected with five different communication networks. In addition to the biological computer controls cell metabolism and function assigned to a given type of cell. Such system permits smooth cooperation between 10¹⁷ cells forming the body. Within a communication system each network utilizes different types of signals, with about 69 different signals operating within each network. These numbers permit the formation of a circa of 10⁹⁹ combinations approaching the infinity and utilizing different types of signals. Practically each cell biological computer can recognize signals delivered by every one in the network.

### CROSS-REFERENCE TO RELATED APPLICATIONS

This application claims benefit of U.S. Provisional Patent Application Ser. No. 61/201,909 filed Dec. 16, 2008, which is incorporated herein by reference in its entirety.

### TECHNICAL FIELD OF THE INVENTION

The present invention relates in general to the protein therapeutics, and more particularly, to the discovery of a proline rich peptide complexes useful in cellular communication mechanisms and in the modulation of diseases including Alzheimer's disease (AD).

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with the uses of colostrinin and colostrinin peptides in the prevention and treatment of diseases.

U.S. Pat. No. 6,500,798 issued to Stanton et al. (2002) provides methods that utilize compositions containing colostrinin, and constituent peptides thereof, an active analog thereof, and combinations thereof, as an oxidative stress regulator. The Stanton patent describes the use of colostrinin, at least one constituent (i.e., component) peptide thereof, at least one active analog thereof (e.g., peptide having an N-terminal sequence equivalent to an N-terminal sequence of at least one of the colostrinin constituent peptides), and combinations thereof, as an oxidative stress regulator. These oxidative stress regulators can be used in vitro or in vivo, including internal and external use in animals including mammals such as humans. They can be used for preventative (i.e., prophylactic) treatments or for therapeutic treatment.

U.S. Pat. No. 7,119,064 issued to Boldogh et al. (2006) describes methods that utilize compositions containing colostrinin, a constituent peptide thereof, an active analog thereof, and combinations thereof, as modulators of intracellular signaling molecules. The '064 patent relates to the use of colostrinin, at least one constituent (i.e., component) peptide thereof, at least one active analog thereof (e.g., peptide having an N-terminal sequence equivalent to an N-terminal sequence of at least one of the colostrinin constituent peptides), and combinations thereof, as modulators of intracellular signaling mechanisms. The signaling molecules discovered to date that are modulated include 4HNE adduct formation, GSH, P53, and JNK.

### SUMMARY OF THE INVENTION

The present invention describes novel communication system governing circa two hundreds of different types of cells in each mammal. It modulates the activities of the organism through the biological computer present in every living cell. Different type of cells can communicate with other through biological computer connected with five different communication networks. In addition to the biological computer controls cell metabolism and function assigned to a given type of cell. Such system permits smooth cooperation between 10¹⁷ cells forming the body. Within a communication system each network utilizes different types of signals. About 69 different signals operate within each network. These numbers permit the formation of a circa of 10⁹⁹ combinations approaching the infinity and utilizing different types of signals. Practically each cell biological computer can recognize signals delivered by every one in the network.

Network I is located in the brain (CN I). It utilizes electric signals + and −. Signal + is the activate reaction. Signal − terminates communication. CN I controls other network activities and represents the largest computer system in the world.

Network II is called Hormonal-Cytokine (CN II). It governs the host organs through signals generated by hormonal and immune networks. The signal is coded in DNA/RNA system. The synthesis of the activation signal requires time to locate where the signal is coded. Next step requires activating the storage, synthesis of the signal, and transfer to the target, where it binds to specific receptors that transfer messages to cell initiating the cellular reactions. The signals from CN II, although very precise, are relatively slow. Their biological role is protecting the host organs against environmental insults (EI).

Network III (CN III) is formed by group of triplets (three amino acids) carrying information encoded on amino acid proline. The triplets are coded in DNA/RNA system. In addition, ready to go forms are expressed in two proteins; β Casein and “PRTB”. The triplets coded in β-casein are species specific and distributed between the 56 and 241 amino acids of the protein (of the given species). The triplet comprises three amino acids where proline plays a crucial role, because it can record the information by changing to cis-trans-cis configuration. Coded information on proline is stabilized by attachment to two satellite peptides (amino acids) with characteristic hydrophobic profiles. This type of the amino acid system coding has infinite memory (20×20×20=8000. 8000!=infinity) and still has a plenty of free space for store future new information. Combination of more than one triplet creates programs used to activate cellular computer at the beginning of cell life. Network CN III is also capable to restore computer function when the EI damages the cell. As of today, the 69 triplets assigned to network create a system with 10⁹⁸ combinations capacity. The system is constantly growing larger by addition newly discovered triplets to the network.

Network IV (CN IV) is formed in very similar fashion as CN III. The difference is in the mode of triplet formation. They were created at time when the cells were in “primordial soup”. At that time the synthesis of peptides and protein was carried by an alternative protein synthesis method. CN IV is considered as the oldest form of communication and collection of primordial information concerning interrelation with environmental factors. Information collected in this system in the Proline amino acids, formed the base for development DNA/RNA system. Triplets formed during this period of time also existed in other species since they were constructed before the species differentiation period. The systems still remain in the human body and control essential cell functions.

Network V (CN V) has a role different from others. Members of this network control amplitude of the reactions induced by EI. Two basic mechanisms govern this system; amplification and inhibition (tachophylaxis) of the signals. Amplification of the signal frequently occurs in the physiological situation when the host protects the cells against incoming pathogens. Classical example is the signal amplification induced by Interferon α. The amplifying agent in this case is Interferon γ. The reverse situation is caused by loss of control over Interferon α production. It occurs during infection with Argentinean Hemorrhagic fever. The invading virus block the natural feed back mechanism controlling the level of Interferon a production. An infected individual start to synthesize such large quantity of IFN a that it frequently kills the host. The triplets present in CN III and CN IV can repair EI damages done to biological computers. The problem is to locate the damages inside cell computer. The best strategy is to deliver to the host all elements potentially needed and permitting the host to utilize those that are needed (for repair). It is not necessary to worry about delivery of useless triplets because the host can utilize them later as source of amino acids or energy. The problem is created in the delivery of biologically active elements to the cells, which require their presence. The triplets in the native form, assigned to repair the damages may easily enter into a reaction with inert elements of the body and be lost (for repair purposes).

To protect such situation the host has developed a system, which delivers needed elements to addressee. The large proteins like γ globulin may play role of carrier. Also there are smaller peptides, with amino acids formulated hydrophobic or hydrophilic regions to which triplets may adsorb and form complexes. Such complexes as inert are delivered to the addressee to release cargo (triplets). Simple changes in pH, elevation of the temperature or ion concentrations of the affected cells, may trigger release of needed triplets. Free carriers may also become scavengers for free radicals or become acceptors for pathogens or its toxic products. Loaded with this type of material they deliver it to antibody producing centers, stimulating the host defenses. The peptide role as the carrier peptides is a new element of innate immunity as discovered by the present inventors. The newly discovered CN III and CN IV range of activity is enormous: the possible combinations of signals approach infinity. The large numbers of signal explain difficulties faced by the pharmaceutical industry focused on a single synthetic drug delivery. By definition majority of drugs are not physiological agents and may interact with the host elements, inducing adverse reactions. Since the area of the interactions, are so large (10⁹⁸) it is very difficult to design a drug to specifically reacting with a selected target. The adverse reactions may be more detrimental than beneficial effect of the drug (interference with many different complexes).

The model of new communication systems presented herein together with it basic functions open new possibility to modulate human health. Three areas are particularly significant: diagnostic, baby food, and human health.

The large area of human life affected by new communication model required identification and location of possible damages, the present invention addresses this problem by providing an answer as to whether single or multiple signals are damaged, whether differences exist in a communication network exists due to differences in race, languages or other variables line EI, whether, the aberrations are quantitative or qualitative occurring due to the age or presence of a disease, etc. Present day baby food while providing the required nutrients and caloric value is inadequate in protecting against EI and fails to deliver the needed information to the natural biologic computers leading to possible developmental defects in babies and in protection against EI. The new discoveries in communication systems as described in the present invention provide an opportunity to manage human health particularly in prophylaxis and therapy management.

The present invention discloses a nutritional composition comprising: one or more antioxidants or one or more free radical scavengers (identical for the purposes of the instant invention) derived from one or more animal or plant peptides, one or more milk peptides, one or more optional naturally occurring constituents, wherein the naturally occurring constituents comprise amino acids, vitamins, oils, fatty acids, pigments, medicinal herbs and fruits, carotenoids, and combinations thereof, and one or more optional inert agents or additives, wherein the inert agents or additives comprise excipients, preservatives, bulking agents, gelatin, coloring agents, and combinations thereof. The composition disclosed in the present invention is used to prevent Alzheimer's disease, vascular dementias, Pick's disease, Creutzfeldt-Jacobs disease, Parkinson's disease, HIV, mad cow disease, and aging. The beneficial effects of the composition disclose herein arises from prevention of the depletion of one or more neurons and stimulates repair of one or more damaged neurons. In one aspect of the present invention the peptide is bovine lactoferrin. In another aspect the one or more milk peptides comprise amino acid triplets, amino acid complexes or both, wherein the amino acid triplets, the amino acid complexes or both comprise proline as one or all of the amino acids triplet or the complex. In yet another aspect the amino acid triplets, amino acid complexes or both are comprised in one or more cellular signaling or communication mechanisms.

The amino acid triplets described herein comprise APV, PIP, APQ, PKV, DPQ, PYG, LPP, QPA, LPI, VPV, MPV, DPK, NPT 3X, VPS 2X, PQP 2X, YPV, PTI, PQQ, and combinations thereof and the amino acid complexes comprise QPQ PLI YPF (SEQ. ID NO.: 46), VPQ PKV LPI PQQ (SEQ. ID NO.: 54), LPQ, YPY PQQ AVP (SEQ. ID NO.: 72), LPL AQP (SEQ. ID NO.: 54), QPL RPV (SEQ. ID NO.: 73), LPV PQP (SEQ. ID NO.: 51), NPI, VPK, PTI PFF DPQ (SEQ. ID NO.: 56), PKL, LPL PLL QPL (SEQ. ID NO.: 50), LPP QPL (SEQ. ID NO.: 49), and combinations thereof. In one aspect the amino acid triplets, the amino acid complexes or both may be derived from human β-casein.

In another aspect the amino acid triplets derived from human β-casein comprise APV, AQP, AVP, DPQ, LPL, LPV, LPP, LPI, MPV, NPT, and combinations thereof. In yet another aspect the amino acid complexes derived from human 13-casein comprise QPQPLIYPF (SEQ ID NO.: 46), VPYPQQAVP (SEQ ID NO.: 52), EPIPYG (SEQ ID NO.: 47), LPLAQP (SEQ ID NO.: 48), QPLAPV (SEQ ID NO.: 53), VPQPKVLPIPQQ (SEQ ID NO.: 54), LPPQPL (SEQ ID NO.: 49), VPQPIP (SEQ ID NO.: 55), LPLPLLQPL (SEQ ID NO.: 50), PTIPFFDPQ (SEQ ID NO.: 56), LPVPQP (SEQ ID NO.: 51), and combinations thereof. Finally, the composition of the present invention is a capsule, a tablet, a powder, a drink, a baby formula, or any suitable orally consumable form.

In one embodiment the present invention describes a composition for preventing Alzheimer's disease comprising:

| | |
|---|---|
| Vitis viniferous oil | 0.1-50%; |
| Anthocyanes isolated from Aronia berries | 0.001-2%; |
| Hyppohae Ramnoides L | 0.01-10%; |
| Bovine Lactoferrin | 0.1-10%; wherein the |
| | bovine lactoferrin is isolated |
| | from animal milk, e.g., |
| | bovine milk; |
| Flax oil | 0.1-10%; |
| Conjugated linoleic acids (C.L.A) | 0.1-10%; |
| Tocoferol acetate | 0.1-10%; |
| Lutein | 1.0-5 mg; |
| Antioxidants and animal milk peptides | 0.0001-10%; |
| P-hydroxybenzoate, 1 | 0-20%; |
| Amorphic silica oxide, 1 | 0-50%; and |
| Fish gelatin to make a 0.5 g capsule. | |

The composition described above is used to prevent vascular dementias, Pick's disease, Creutzfeldt-Jacobs disease, Parkinson's disease, HIV, mad cow disease, and premature aging. In one aspect the one or more milk peptides comprise amino acid triplets, amino acid complexes or both. In another aspect the amino acid triplets, the amino acid complexes or both comprise proline as one or all of the amino acids triplet or the complex, wherein the amino acid triplets, amino acid complexes or both are comprised in one or more cellular signaling or communication mechanisms. In yet another aspect the amino acid triplets comprise PKV, PYG, NPT 3X, PQP 2X, and combinations thereof and the amino acid complexes comprise QPQ PLI YPF (SEQ ID NO.: 46), VPQ PKV LPI PQQ (SEQ ID NO.: 54), LPQ, YPY PQQ AVP (SEQ ID NO.: 72), LPL AQP (SEQ. ID NO.: 48), QPL RPV (SEQ. ID NO.: 73), LPV PQP (SEQ. ID NO.: 51), NPI, PTI PFF DPQ (SEQ. ID NO.: 56), PKL, LPL PLL QPL (SEQ. ID NO.: 50) and combinations thereof.

In yet another aspect the amino acid triplets comprise APV, PIP, APQ, PKV, DPQ, PYG, LPP, QPA, LPI, VPV, MPV, DPK, NPT 3X, VPS 2X, PQP 2X, YPV, PTI, PQQ, APP, CPP, FPG, GPI, HPG, LPM, NPV, PPG, PPP, RPS, QPT, VPT 2X, YPV, YPQ, FPE, IPN, LPL, LPV, LPQ, MPI, PLL, PKY, PFT, PIL, PQR, QPL 3X, VPQ, VPF, VPP, VPY, VPV, APV, APQ, DPQ, LPP, LPI, MPV, NPT 3X, PTI, PQQ, PIP, PKV, QPA, VPV, DPK, YPV, PYG, VPS 2X, LPQ 2X, QPP 2X, PKL, NPI, and combinations thereof and the amino acid complexes comprise QPQ PLI YPF (SEQ ID NO.: 46), VPQ PKV LPI PQQ (SEQ ID NO.: 54), LPQ, YPY PQQ AVP (SEQ ID NO.: 72), LPL AQP (SEQ. ID NO.: 48), QPL RPV (SEQ. ID NO.: 73), LPV PQP (SEQ. ID NO.: 51), NPI, VPK, PTI PFF DPQ (SEQ. ID NO.: 56), PKL, LPL PLL QPL (SEQ. ID NO.: 50), LPP QPL 3X (SEQ. ID NO.: 49), YPT QPT YPV QPP (SEQ. ID NO.: 67), NPV YPQ (SEQ. ID NO.: 68), LPQ APP (SEQ. ID NO.: 69), YPV QPI YPP (SEQ. ID NO.: 70), PPP PPG CPP (SEQ. ID NO.: 71), VPK, and combinations thereof.

In one aspect the amino acid triplets, the amino acid complexes or both may be derived from β-casein comprising proline as one or all of the amino acids triplet or the complex. In another aspect the amino acid triplets derived from β-casein comprise LPL, LPV, NPT, and combinations thereof. In yet another aspect the amino acid complexes derived from β-casein comprise VPYPQQAVP (SEQ ID NO.: 52), EPIPYG (SEQ ID NO.: 47), and combinations thereof. The composition of the present invention is administered once daily. The triplets and peptides mentioned above are of animal origin and serve as scavengers of free radicals protecting neurons from oxidative stress damages and against apoptosis.

In one aspect the amino acid triplets, the amino acid complexes or both may be derived from β-casein comprising proline as one or all of the amino acids triplet or the complex. In another aspect the amino acid triplets derived from β-casein comprise APV, AQP, AVP, DPQ, LPL, LPV, LPP, LPI, MPV, NPT, and combinations thereof. In yet another aspect the amino acid complexes derived from β-casein comprise QPQPLIYPF (SEQ ID NO.: 46), VPYPQQAVP (SEQ ID NO.: 52), EPIPYG (SEQ ID NO.: 47), LPLAQP (SEQ ID NO.: 48), QPLAPV (SEQ ID NO.: 53), VPQPKVLPIPQQ (SEQ ID NO.: 54), LPPQPL (SEQ ID NO.: 49), VPQPIP (SEQ ID NO.: 55), LPLPLLQPL (SEQ ID NO.: 50), PTIPFFDPQ (SEQ ID NO.: 56), LPVPQP (SEQ ID NO.: 51), and combinations thereof. The composition of the present invention is administered once daily. The triplets and peptides mentioned above are of animal origin and serve as scavengers of free radicals protecting neurons from oxidative stress damages and against apoptosis.

In another embodiment the present invention provides a method of preventing Alzheimer's Disease (AD) in a human subject comprising the steps of: (i) identifying the human subject in need of prevention of AD and (ii) administering a composition once daily to the human subject for the prevention of AD, wherein the composition comprises one or more antioxidants, one or more free radical scavengers derived from animal or plant peptides, one or more milk peptides, and one or more optional amino acids, vitamins, oils, fatty acids, pigments, medicinal herbs and fruits, carotenoids, inert agents, additives, and combinations thereof. In one aspect the one or more milk peptides comprise amino acid triplets, amino acid complexes or both, wherein the amino acid triplets, the amino acid complexes or both comprise proline as one or all of the amino acids triplet or the complex. In another aspect the amino acid triplets, amino acid complexes or both are comprised in one or more cellular signaling or communication mechanisms. In yet another aspect the amino acid triplets, the amino acid complexes or both may be derived from animal β-casein.

The present invention further discloses a nutritional composition comprising: one or more antioxidants, derived from one or more animal or plant peptides, one or more synthetic or natural milk peptides, wherein the peptides comprise one or more amino acid triplets, one or more amino acid complexes or both, one or more optional naturally occurring constituents, wherein the naturally occurring constituents comprise amino acids, vitamins, oils, fatty acids, pigments, medicinal herbs and fruits, carotenoids, and combinations thereof, and one or more optional inert agents or additives, wherein the inert agents or additives comprise excipients, preservatives, bulking agents, gelatin, coloring agents, and combinations thereof. In one aspect the composition is used to ameliorate symptoms Alzheimer's disease (AD), vascular dementias, Pick's disease, Creutzfeldt-Jacobs disease, Parkinson's disease, HIV, mad cow disease, and aging. In another aspect the composition repairs one or more damaged neurons and prevents damage induced by one or more free radicals. In yet another aspect the peptide is bovine lactoferrin.

In one aspect the composition of the present invention comprises amino acid triplets, the amino acid complexes or both comprising proline as one or all of the amino acids triplet or the complex comprised in one or more cellular signaling or communication mechanisms. The amino acid triplets comprise, peptides from the Protein Rich Triplet-Brain (PRTB) complex, APP, CPP, FPG, GPI, HPG, LPM, NPV, PPG, PPP, RPS, QPT, VPT 2X, YPV, YPQ, FPE, IPN, LPL, LPV, LPQ, MPI, PLL, PKY, PFT, PIL, PQR, QPL 3X, VPQ, VPF, VPP, VPY, VPV, APV, APQ, DPQ, LPP, LPI, MPV, NPT 3X, PTI, PQQ, PIP, PKV, QPA, VPV, DPK, YPV, PYG, VPS 2X, LPQ 2X, QPP 2X, PKL, NPI, and combinations thereof. The amino acid complexes comprise YPT QPT YPV QPP (SEQ. ID NO.: 67), NPV YPQ (SEQ. ID NO.: 68), LPQ APP (SEQ. ID NO.: 69), YPV QPI YPP (SEQ. ID NO.: 70), PPP PPG CPP (SEQ. ID NO.: 71), LPL PLL QPL (SEQ. ID NO.: 50), LPP QPL 3X (SEQ. ID NO.: 49), VPQ PKV LPI PQQ (SEQ. ID NO.: 54), YPY PQQ AVP (SEQ. ID NO.: 72), QPL RPV (SEQ. ID NO.: 73), QPQ PLI YPF (SEQ. ID NO.: 46), LPQ, LPL AQP (SEQ. ID NO.: 48), LPV PQP (SEQ. ID NO.: 51), VPK, PTI PFF DPQ (SEQ. ID NO.: 56), and combinations thereof, wherein the sequences are derived from human species. Finally, in another aspect the composition is a capsule, a tablet, a powder, a drink, a baby formula, or any suitable orally consumable form.

In a specific embodiment the present invention discloses a composition for mitigating the symptoms of Alzheimer's disease (AD) comprising:

| | |
|---|---|
| Vitis viniferous oil | 0.1-50%; |
| Anthocyanes isolated from Aronia berries | 0.001-2%; |
| Hyppohae Ramnoides L | 0.01-10%; |
| Bovine Lactoferrin | 0.1-10%; wherein the |
| | bovine lactoferrin is isolated |
| | from bovine milk; |
| Flax oil | 0.1-10%; |
| C.L.A | 0.1-10%; |
| Tocoferol acetate | 0.1-10%, |
| Lutein | 1.0-5 mg; |

Synthetic or milk peptides in an amount sufficient to treat or prevent the symptoms of AD, wherein the peptides comprise one or more amino acid triplets, one or more amino acid complexes or both;

| | |
|---|---|
| P-hydroxybenzoate, 1 | 0-20%; |
| Amorphic silica oxide, 1 | 0-50%; and |
| Fish gelatin to make a 0.5 g capsule. | |

The amino acid triplets described herein comprise APV, PIP, APQ, PKV, DPQ, PYG, LPP, QPA, LPI, VPV, MPV, DPK, NPT 3X, VPS 2X, PQP 2X, YPV, PTI, PQQ, and combinations thereof and the amino acid complexes comprise QPQ PLI YPF (SEQ ID NO.: 46), VPQ PKV LPI PQQ (SEQ ID NO.: 54), LPQ, YPY PQQ AVP (SEQ ID NO.: 72), LPL AQP (SEQ. ID NO.: 48), QPL RPV (SEQ. ID NO.: 73), LPV PQP (SEQ. ID NO.: 51), NPI, VPK, PTI PFF DPQ (SEQ. ID NO.: 56), PKL, LPL PLL QPL (SEQ. ID NO.: 50), LPP QPL (SEQ. ID NO.: 49), and combinations thereof. In one aspect the amino acid triplets, the amino acid complexes or both may be derived from human β-casein.

In another aspect the amino acid triplets derived from human β-casein comprise APV, AQP, AVP, DPQ, LPL, LPV, LPP, LPI, MPV, NPT, and combinations thereof. In yet another aspect the amino acid complexes derived from human β-casein comprise QPQPLIYPF (SEQ ID NO.: 46), VPYPQQAVP (SEQ ID NO.: 52), EPIPYG (SEQ ID NO.: 47), LPLAQP (SEQ ID NO.: 48), QPLAPV (SEQ ID NO.: 53), VPQPKVLPIPQQ (SEQ ID NO.: 54), LPPQPL (SEQ ID NO.: 49), VPQPIP (SEQ ID NO.: 55), LPLPLLQPL (SEQ ID NO.: 50), PTIPFFDPQ (SEQ ID NO.: 56), LPVPQP (SEQ ID NO.: 51), and combinations thereof. Finally, the composition of the present invention is a capsule, a tablet, a powder, a drink, a baby formula, or any suitable orally consumable form.

In one aspect the composition is used to treat or mitigate the symptoms of vascular dementias, Pick's disease, Creutzfeldt-Jacobs disease, Parkinson's disease, HIV, mad cow disease, and aging. In another aspect the amino acid triplets, the amino acid complexes or both comprise proline as one or all of the amino acids triplet or the complex. In yet another aspect the amino acid triplets, amino acid complexes or both are comprised in one or more cellular signaling or communication mechanisms. The amino acid triplets as described in the present invention comprise, peptides from the PRTB complex, APP, CPP, FPG, GPI, HPG, LPM, NPV, PPG, PPP, RPS, QPT, VPT 2X, YPV, YPQ, FPE, IPN, LPL, LPV, LPQ, MPI, PLL, PKY, PFT, PIL, PQR, QPL 3X, VPQ, VPF, VPP, VPY, VPV, APV, APQ, DPQ, LPP, LPI, MPV, NPT 3X, PTI, PQQ, PIP, PKV, QPA, VPV, DPK, YPV, PYG, VPS 2X, LPQ 2X, QPP 2X, PKL, NPI, and combinations thereof. The amino acid complexes comprise YPT QPT YPV QPP (SEQ. ID NO.: 67), NPV YPQ (SEQ. ID NO.: 68), LPQ APP (SEQ. ID NO.: 69), YPV QPI YPP (SEQ. ID NO.: 70), PPP PPG CPP (SEQ. ID NO.: 71), LPL PLL QPL (SEQ. ID NO.: 50), LPP QPL (SEQ. ID NO.: 49), VPQ PKV LPI PQQ (SEQ. ID NO.: 54), YPY PQQ AVP (SEQ. ID NO.: 72), QPL RPV (SEQ. ID NO.: 73), QPQ PLI YPF (SEQ. ID NO.: 46), LPQ, LPL AQP (SEQ. ID NO.: 48), LPV PQP (SEQ. ID NO.: 51), VPK, PTI PFF DPQ (SEQ. ID NO.: 56), and combinations thereof, wherein the sequences are derived from human species. The composition of the present invention is administered once daily.

In yet another embodiment the present invention is describes a method of mitigating the symptoms of Alzheimer's Disease (AD) in a human subject comprising the steps of: identifying the human subject in need of mitigation of the symptoms of the AD and administering a composition once daily to the human subject in a quantity sufficient for the mitigation of the symptoms of the AD, wherein the composition comprises one or more antioxidants, (one or more free radical scavengers) derived from animal or plant peptides, one or more synthetic or milk peptides comprising amino acids triplets, amino acid complexes or both, and one or more optional amino acids, vitamins, oils, fatty acids, pigments, medicinal herbs and fruits, carotenoids, inert agents, additives, and combinations thereof. The method of the present invention further comprises the steps of: monitoring the progression of the treatment by measuring a cognitive state of the human subject at regular intervals by performing a mini mental state examination (MMSE) and other test scores used for an evaluation of cognitive state of the human subject and altering the quantity of the composition administered or stopping the administration of the composition based on a result of the MMSE and other test scores used for the evaluation of the cognitive state of the human subject. In one aspect the amino acid triplets, the amino acid complexes or both comprise proline as one or all of the amino acids triplet or the complex. In another aspect the amino acid triplets, amino acid complexes or both are comprised in one or more cellular signaling or communication mechanisms. In yet another aspect the amino acid triplets comprise, peptides from the PRTB complex, APP, CPP, FPG, GPI, HPG, LPM, NPV, PPG, PPP, RPS, QPT, VPT 2X, YPV, YPQ, FPE, IPN, LPL, LPV, LPQ, MPI, PLL, PKY, PFT, PIL, PQR, QPL 3X, VPQ, VPF, VPP, VPY, VPV, APV, APQ, DPQ, LPP, LPI, MPV, NPT 3X, PTI, PQQ, PIP, PKV, QPA, VPV, DPK, YPV, PYG, VPS 2X, LPQ 2X, QPP 2X, PKL, NPI, and combinations thereof. Finally the amino acid complexes comprise YPT QPT YPV QPP (SEQ. ID NO.: 67), NPV YPQ (SEQ. ID NO.: 68), LPQ APP (SEQ. ID NO.: 69), YPV QPI YPP (SEQ. ID NO.: 70), PPP PPG CPP (SEQ. ID NO.: 71), LPL PLL QPL (SEQ. ID NO.: 50), LPP QPL (SEQ. ID NO.: 49), VPQ PKV LPI PQQ (SEQ. ID NO.: 54), YPY PQQ AVP (SEQ. ID NO.: 72), QPL RPV (SEQ. ID NO.: 73), QPQ PLI YPF (SEQ. ID NO.: 46), LPQ, LPL AQP (SEQ. ID NO.: 48), LPV PQP (SEQ. ID NO.: 51), VPK, PTI PFF DPQ (SEQ. ID NO.: 56), and the combinations thereof. The triplets and short peptides mentioned above are all present in human CN III. They help new born neurons to program their biological computers. Only if it is programmed in this fashion can the new neuron replace the lost or damaged neuron.

In one embodiment the present invention is an immunological method for the determination of a titer of one or more triplets, peptides, proline rich peptide complexes (PRPCs) in a colostrum or other biological fluids comprising the steps of: (i) providing one or more monospecific antibodies against the one or more amino acids, the triplets, wherein the monospecific antibodies are raised in a rabbit, a horse, a sheep or a goat by injecting the one or more amino acids, the triplets, or the PRPCs, wherein the one or more amino acids, triplets, the PRPCs, may be conjugated with a polymer or non-immunogenic amino acids (complex) or peptides, (ii) determining a cross-reactivity of the one or more monospecific antibodies with the one or more amino acids, the triplets, the peptides, the PRPCs in an Enzyme Linked Immuno-adsorbent Assay (ELISA), and (iii) determining the titer for a specific amino acid of the one or more amino acids, the triplets, the PRPCs based on the extent of cross-reactivity between the monospecific antibodies and the one or more amino acids, triplets, the PRPCs. In one aspect of the method the peptides comprises 1, 2, 3, 4, 5, 7, 8, 9, 10, 11, 12, 13, 14, 15, 16, 17, 18, 19, 20 or more of the peptides selected from LQTPQPLLQVMMEPQGD (SEQ. ID NO.: 1), MPQNFYKLPQM (SEQ. ID NO.: 2), VLEMKFPPPPQETVT (SEQ. ID NO.: 3), LKPFPKLKVEVFPFP (SEQ. ID NO.: 4), SEQP (SEQ. ID NO.: 5), DPPPPQS (SEQ. ID NO.: 6), MIVVRLLQNEVPE (SEQ. ID NO.: 7), SLSQSKVLPV (SEQ. ID NO.: 8), LQTQTPVV (SEQ. ID NO.: 9), QPLLQVMMEPQ (SEQ. ID NO.: 10), VESYVPLFP (SEQ. ID NO.: 11), LPPNVG (SEQ. ID NO.: 12), SEEMP (SEQ. ID NO.: 13), DSQPPV (SEQ. ID NO.: 14), FPPPK (SEQ. ID NO.: 15), DLEMPVLPVEPFPFV (SEQ. ID NO.: 16), LFFFLPVVNVLP (SEQ. ID NO.: 17), MQPPPLP (SEQ. ID NO.: 18), DQPPDVEKPDLQPFQVQS (SEQ. ID NO.: 19), LVYPFTGPIPNSLPQNILP (SEQ. ID NO.: 20), EMPFPKY (SEQ. ID NO.: 21), and 1 Lacto 1.

### BRIEF DESCRIPTION OF THE DRAWINGS

For a more complete understanding of the features and advantages of the present invention, reference is now made to the detailed description of the invention along with the accompanying figures and in which:

FIGS. 1A-1AC show the hydrophobicity profiles of simple triplets: (A) EPQ, (B) EMP, (C) RPD, (D) RPK, (E) PNS, (F) PQS, (G) PQK, (H) TPQ, (I) TPG, (J) QPP, (K) PEV, (L) PPV, (M) EPF, (N) HPI, (O) PLT, (P) MPQ, (Q) VPE, (R) LKP, (S) YPF, (T) LPV, (U) LPL, (V) PLL, (W) PFV, (X) PKV, (Y) TPV, (Z) VLP, (AA) VFP, (AB) FPP, and (AC) EVE;

FIGS. 2A-2U show the hydrophobicity profiles of complex triplets: (A) QPPD (SEQ. ID NO.: 74), (B) QPPQ (SEQ. ID NO.: 75), (C) QPPV (SEQ. ID NO.: 76), (D) QPPF (SEQ. ID NO.: 77), (E) VPPF (SEQ. ID NO.: 78), (F) FPPQ (SEQ. ID NO.: 38), (G) FPPPK (SEQ. ID NO.: 15), (H) KPFPK (SEQ. ID NO.: 79), (I) GPIPN (SEQ. ID NO.: 80), (J) EPFPF (SEQ. ID NO.: 81), (K) YPFPI (SEQ. ID NO.: 82), (L) DPPPQ (SEQ. ID NO.: 83), (M) QPPPQ (SEQ. ID NO.: 84), (N) FPFPF (SEQ. ID NO.: 85), (O) VPLFP (SEQ. ID NO.: 86), (P) GPPF (SEQ. ID NO.: 87), (Q) KPFPK (SEQ. ID NO.: 79), (R) FPPPQ (SEQ. ID NO.: 88), (S) TPQPL (SEQ. ID NO.: 89), (T) EPF, and (U) FPK; and

FIGS. 3A-3D is a plot showing the results of the Minimal Mental State Examination (MMSE) of AD patients following administration of Bio-6 measured at bi-monthly intervals: (A) MMSE=20-24, (B) MMSE=18-19, (C) MMSE=10-12, and (D) MMSE<9.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

It is contemplated that any embodiment discussed in this specification can be implemented with respect to any method, kit, reagent, or composition of the invention, and vice versa. Furthermore, compositions of the invention can be used to achieve methods of the invention.

The present invention describes a new model of communication system operating in a mammalian species. The information concerning control of the metabolic processes and repair damages were transferred to progeny in form of communication network. Theory assumes presence of five communication networks which at different level, controls function of organism consisted from circa 200 cells forming the body having 10¹⁶ to 10¹⁷ cells. To satisfy demand of the host, the network is using different type of signals carrying different set of information coded on different carriers and addressed to different part of the organisms. A similar organization of communication systems is present in humans and other mammalian species.

The communication systems comprise of five different networks as stated previously that operate by means of different signals. The oldest network appeared with the first born cell in primordial soup circa two billion years ago. Network I is located in the brain (CN I) and represents the largest computer system in the world. Network II is called Hormonal-Cytokine (CN II). It governs the host organs through hormonal and immune networks. The present invention describes three additional and novel communication systems designated as CN III, CN IV, and CN V. The novel networks of the present invention comprise:

Network III (CN III) is formed by group of triplets (three amino acids) carrying information encoded on amino acid proline. The triplets are coded in the DNA of the DNA/RNA system. In addition, ready to go forms are expressed in two proteins; β Casein and Protein Rich Triplet-Brain “PRTB”.

Network IV (CN IV) is formed in very similar fashion as CN III. Is the oldest and uses as signals the triplets comprising proline, where proline acts as a carrier of the holographic memory started process of collecting information of what the ancestor cells did to survive insults from environment.

Network V (CN V) has a role different from others. Members of this network control amplitude of the reactions induced by EI. Two basic mechanisms govern this system; amplification and inhibition of the signals.

To preserve the information written on proline amino acid cis-trans-cis configurations, the host decided to add to proline two different amino acids with particular hydrophobicity profile. Thus, creating a system of unlimited possibilities. 8000 triplets can create unlimited number of information. The cis-trans-cis configuration stabilized by satellite peptides begins the formation of the memory bank that permits the further cellular development and creation (for example. DNA/RNA system). At the same time information is provided to control basic metabolic processes and protect cells, tissue and the host against the free radicals.

There are two types of free radicals that exist: (i) from electrons and (ii) from amino acid, peptides and other metabolite (compounds), that operates within the host and lacking an electron from their orbit. As a consequence the host has to be protected by bunch of electrons and “tabunes” or cohorts of molecules ready to interact with everything, that have free electron on their surface. This cohort is normally under the control of enzymes and are utilized by enzyme in the metabolism providing the cell with needed elements to function properly. The situation is further complicated by the addition of inducers of free radicals in various EI: for, e.g., germs, spirochete, Rickettsiae, molds, yeast, Mycoplasmas, Viruses and prions. The host is then faced with a deadly mixture of EI.+oxygen, temperature and light.

With the time and phyllogenetic forces the host starts to deviate from brothers and sisters forming new races, species resulting in a genetic mix up. The new species started to collect their own information coded on the same types of triplet but different from their cousins.

During the birth the embryos repeat the phase, similar to ancestors, where they abandon the water environment and initiate life on dry land. This step is accompanied by a powerful oxidative stress, generated by differences in concentration of oxygen in air and mother's womb. Armed with their own RNA/DNA system to start life on dry land the species had to overcome problem with concentration of oxygen, a main sources of free radicals. To combat this they developed an antioxidant strategy comprising of antioxidant, scavengers. This whole program is coded on triplets that had to be delivered to the offspring immediately after the birth. The anti oxidant-scavengers formed base for CN III and other antioxidants of animal and plant origin served to help in the process. The new network operates using the same principal signals as CN IV but information was coded additionally in DNA and stored in a DNA/RNA facility. The host retains the old method and uses it together with the new one. Both are still very useful until now.

The stress is neutralized by species specific Proline Rich Peptide Complex (PRPC). The newborn at the birth remains technically as an embryo and as such have several functions protected. These precautionary steps prevent possible rejection the offspring by immune system of mother. To initiate further development, newborn requires immediate activation dormant cells, organs and inhibited metabolism. Mother's help is provided in the colostrums and milk. At that stage, mother delivers also the programs essential for activation communication network (CN). The information for activation cells biological computers has to be species specific for harmonious development. The programs collected by millennia facilitate the baby's adaptation to the life on dry land; activate the adult metabolism and development the defense system. With the help of mother informers-chaperones initiate multi dimensional processes leading to activation communication network and build metabolic processes.

The proline rich peptides of the present invention are informers chaperone or carriers that are coded either in DNA or made by alternative method. Before maturation of the offspring the information is transferred (stored in) to the “Adult Stem Cells” (ASC). Its origin is the mammary gland of mother. During lactation period the stem cells are released (from the mammary glands) to colostrums and milk (together with the information needed by the offspring). Those groups of peptides coded by called proline rich complex (PRPC) are delivered to be implanted into the offspring (with the colostrums and milk). Stem cells carry the ancestor's wisdom essential for transformation, maturation, and development (the cells born in offspring). This property permits utilization of ASC products to carry reasonability of colostrums after weaning Released chaperones by the individual, stem cells are not affected by histocompatibility complex.

The PRPC deliver the information through battery of epigenetic regulatory molecule that modifies response to changes in cell's micro environment. The natures of those factors are still fragmentarily known.

Proline Rich Peptides Complex (PRPC): Colostrums and the milk carry group of small peptides circa 300-6000 Daltons. Initially they were considered as the end products of larger protein, cleaved during metabolic processes. This view was modified when proline rich peptide were isolated from ovine colostrums by Janusz, Francek and Lisowski, Wroclaw, Poland in 1974. The fraction of peptides isolated after prolong separation procedure was considered as a single entity. This belief was abandoned when 71 peptides were discovered from single electrophoretic band. Among those, 46 carried proline, whereas 25 remain proline free. The peptides with proline were identified as carriers of ancestor's wisdom. The second group represents carriers of non specific properties useful sources of energy, and amino acids. This proline less group was eliminated at this stage for further consideration. The list of identified in ovine colostrums peptides are divided into six groups as described below:

Group A: LQTPQPLLQVMMEPQGD (SEQ. ID NO.: 1), MPQNFYKLPQM (SEQ. ID NO.: 2), VLEMKFPPPPQETVT (SEQ. ID NO.: 3), LKPFPKLKVEVFPFP (SEQ. ID NO.: 4), SEQP (SEQ. ID NO.: 5), DPPPPQS (SEQ. ID NO.: 6), MIVVRLLQNEVPE (SEQ. ID NO.: 7), SLSQSKVLPV (SEQ. ID NO.: 8), LQTQTPVV (SEQ. ID NO.: 9), and QPLLQVMMEPQ (SEQ. ID NO.: 10). Two types of peptides were recognized; one having proline in form of multiple repeats (SEQ. ID NOS.: 3 and 6), and second having proline randomly distributed thought the peptide amino acid sequences (SEQ. ID NOS.: 1 and 4). This peculiar property suggests that they represent two types of the information carriers coded at different period of time. Since amount of information coded on peptide depend upon number of proline residue in the peptide, the first group may represent holders of the whole program. They are formulated before development DNA/RNA system.

Group B: VESYVPLFP (SEQ. ID NO.: 11) LPPNVG (SEQ. ID NO.: 12), SEEMP (SEQ. ID NO.: 13), DSQPPV (SEQ. ID NO.: 14), FPPPK (SEQ. ID NO.: 15), DLEMPVLPVEPFPFV (SEQ. ID NO.: 16), LFFFLPVVNVLP (SEQ. ID NO.: 17), MQPPPLP (SEQ. ID NO.: 18), DQPPDVEKPDLQPFQVQS (SEQ. ID NO.: 19), LVYPFTGPIPNSLPQNILP (SEQ. ID NO.: 20), and EMPFPKY (SEQ. ID NO.: 23). The multi proline repeats are formed only in two peptides. They are present in SEQ. ID NOS.: 15 and 18. In addition, three peptides (SEQ. ID NOS.: 12, 14 and 19) are carrying two proline AA.

Group C: VYPFPGPIN (SEQ. ID NO.: 24), SPSLPQNIL (SEQ. ID NO.: 25), TQTPVVVPPF (SEQ. ID NO.: 26), LQPEIMGVPKVKETMVPK (SEQ. ID NO.: 27), HKEMPFPKYPVEPFTESQ (SEQ. ID NO.: 28), SLTLTDVEKLHLPLPLVQ (SEQ. ID NO.: 29), SWMHQPPQLP (SEQ. ID NO.: 30), MHQPPQPLPPTVMF (SEQ. ID NO.: 31), QPLPPTVMFP (SEQ. ID NO.: 32), PQSVH (SEQ. ID NO.: 33), LSQPKVLPVPQKAVPKPDMPIQ (SEQ. ID NO.: 34), RGPFPILV (SEQ. ID NO.: 35), VPPFLQ (SEQ. ID NO.: 36), PKVK (SEQ. ID NO.: 37), FPPQ (SEQ. ID NO.: 38), and PVLGPV (SEQ. ID NO.: 39). This group has no multiple proline repeats peptides. Only proline duplets were found in: SEQ. ID NOS.: 26, 30, 31, 36 and 38. This subgroup represents more recent products that utilize more precise function (enrichment of the language) than one found in previous proline peptides groups. The reason for this type of interpretation is based upon the fact that primordial cell did not had developed DNA system.

Group D: AFLLYQEPVLGPV (SEQ. ID NO.: 40), PVEPFT (SEQ. ID NO.: 41), PMFLQ (SEQ. ID NO.: 42), VQPF (SEQ. ID NO.: 43), RGPTPILV (SEQ. ID NO.: 44), and PVLGPV (SEQ. ID NO.: 45). One of the members RGPTPILV (SEQ. ID NO.: 44) have powerful scavenger property.

Group E: FPP and PPV. The two triplets having double proline (EPP and PPV) represent an interesting finding the triplets with double proline modulate the blood pressure

Group F: RPKHPIKHQ (SEQ. ID NO.: 45). The Precursor for this Nona peptide is alpha S-1 casein. The F-1 peptide has ability to control the cations level in the blood, and the spinal cord fluids. Since the divalent cations like Zn++, Cu++, and Mo++ play an important role in the Alzheimer, Parkinson's and Willmes disease this peptide may have a biological significance in the treatment of these diseases.

The peptides listed above are informers belonging to different networks they are ovine species specific carrying a pool of information to organs and other to cells and play a second role anti scavenger activity. Some peptides from C group, SEQ. ID NOS.: 24, 25, 28, and 34 act as powerful free radicals scavengers with activity stronger than known antioxidants. Due to their size they do not activate processes leading to rejection cells or organs. This is clear advantage over Stem cells that carries histocompatibility antigens on their surface and therefore can't be use in various individuals.

Mammalian PRPC are peptides (e.g., ovine, bovine or others), capable of preventing senile plaque formation and even dissolving the ones that are formed. These peptides have free radical scavenging property, which have non species specific property and therefore can be used by other species.

Wilusz and Polanowski's Nona peptide F-1, on the other hand, facilitate the flow the metal ion in brain.¹ This property is particularly important for AD and Parkinson's disease patients with their metabolic defects. These rare properties are apparently connected with ability to bind transition metals. The Histidine amino acid, having capability to bind divalent cations and therefore may play a crucial role. Hemochromatosis and Wilma patients will also be affected. Application metal binding triplets or peptides may provide relief to them.(Lactoferrin free of Iron ions)

The discovery that the body language, in form of PRPC, fills the gap formed between two earlier discovered communication networks; the brain CN I and Cytokine-hormone CN II. The CN III, CN IV, and CN V filed the gaps between the first cells and CN II. The PRPC provides opportunity to form number of combinations that will eliminate need to use of the prevailing toxic and non-specific drugs. Moreover, they permit a correction of metabolic errors induced by the environmental factors. The common elements of different PRPCs are its anti-oxidant property and the ability to bind and neutralize free radicals in non specific manner.

The present invention describes six groups of proline rich peptides isolated from ovine colostrums. Only peptides listed in group C are coded in DNA and included in beta casein. Remaining peptides shown in sub-groups A, B, D and F are not present in the Genomic Data of amino acids.

The present invention provides evidence that the living organism protects and utilizes information stored in informer peptides (the Alphabet PRPC). The informer-peptide carries species specific information provided by mother and the stem cells. Originally stem cells are present in the mother's mammary gland and delivered to offspring with colostrums and milk. The information was selected by ancestors for the offspring, to facilitate their early life. The human and animals species keep that information to feed the CN and repair mechanism of the host. After weaning, during the rest of the life span, CN III, CN IV and CN V receive peptides from the stem cells.

Before their use the information is stored in type II storage facilities located on the macromolecules. Each species of macromolecules have hydrophilic and hydrophobic regions like i.e. gamma globulins. The triplets, highly charged attach to macromolecule surface and remain protected until the time when they find right place where they are needed. The system fills the time gap between emergency demand and synthesis new generation of peptides-informers. Programming new peptides depends upon information delivered by the complex. The computer programming the new cells, which replace used or dead cells, represents a continuous process occurring in the body from the birth to the host death. This newly discovered mechanism represents key phenomenon that permits the host to program population of short life spun cells as well as cells operating in gland and organs including the host communication.

The present invention describes species specific communication systems operating in mammals within the host body that operate through proline rich peptides forming PRPC present in colostrums and milk. The information is based on the ancestor's wisdom collected from phyllogenetic development of the species. Since colostrums and milk are produced also by other species of mammals it is conceivable to assume the same principle operates among other species either. Applications the PRPC opens a host of new possibilities to modulate the living organism by extending the period free of illnesses and extend the life span.

When aging becomes noticeable and stem cells fail to deliver information peptides, application the PRPC isolated from colostrums and milk will slow down senescence process and extend the individual life spun. Furthermore, it opens possibility to arrest mutation processes that may lead to development of neoplasia.

As discussed hereinabove, the host governs a circa of 200 different cells and has 201 different computers; peripheral or cellular and central one. The only possible communication between the cells is through computers, not through the nerves or large proteins. The computers receive programs in form of triplets. Defects in amino acid supply are important especially for newborns but amino acids alone are not capable to deliver the order/programs. They can only generate circa 2.432×10¹⁸ signals combinations whereas the host needs 10⁹⁹ signals to operate. Thus, there exists quite a large difference in number of signals, thus indicating why the triplets are so important, and not just amino acids. Mother by delivery of colostrums and milk rescues the offspring from the trouble after the birth (oxidative stresses). Fraction rich with proline present in colostrums provide programs to 201 host computers and eliminate oxidative stress.

Triplets as mentioned previously were identified as basic units carrying the information in newly discovered communication networks CN III, CN IV and CN V. In colostrums and milk of mother are present nutrients and species specific information elements essential for the offspring growth. The colostrums and the milk secure the offspring's needs for energy, row materials, and ancestors experiences. Complex CN IV was differentiated from peptides of CN III because they had no precursor protein in the Data Bank. CN-IV is therefore older than CN III and was made by much more primitive method than the former. In ovine colostrums the complex CN IV is formed from four taxonomic sub-groups of peptides.

There are additional triplets connected with alpha 1s casein and PRTB protein. The triplets are called the words comprising informers consist of ready for action molecules. The triplet's alphabets have capacity to carry information needed for cells to control its internal metabolism and communicate with other neighboring cells. The ovine PRPC is formed from 29 complex peptides and several single triplets. They are apparently responsible for securing the host with all what is necessary for correct cellular metabolism.

Chronic diseases develop in two phases. I. initial non-specific manifestation produces large quantities of free radicals. The free radicals damage the different parts of cellular computers inducing specific changes for the given diseases. II. The second phase can be controlled by either of two groups of triplets, (belonging to CN III and CN IV) which are made by two different methods, and stored in two different storage facilities suggesting, that chronic degenerative disorders and chronic infectious processes represent two classes of disorders that require application of two different sets of triplets. Malfunctions in the native immunity leads to the development chronic infectious diseases like Tuberculosis, Leprosies, Herpes, Hepatitis B, C, HIV, and others. The malfunctions described above can occur within CN II or system modulating network called CN III coded in DNA as PRPC 1. The present inventors studied the triplets by analysis of the triplet's functions using their hydrophobicity profile. For example, in hypertension disease, the key element responsible for high blood pressure is enzyme ACE. During the life span of a person with a hypertensive disease, to control enzyme activity, current treatments use a combination of anti hypertensive drugs, e.g., angiotensin-converting enzyme (ACE) inhibitors. One of the ACE inhibitors is the Lisinopril. This triplet acting alone fails to control effectively high blood pressure, but in combination with other drugs it becomes very useful.

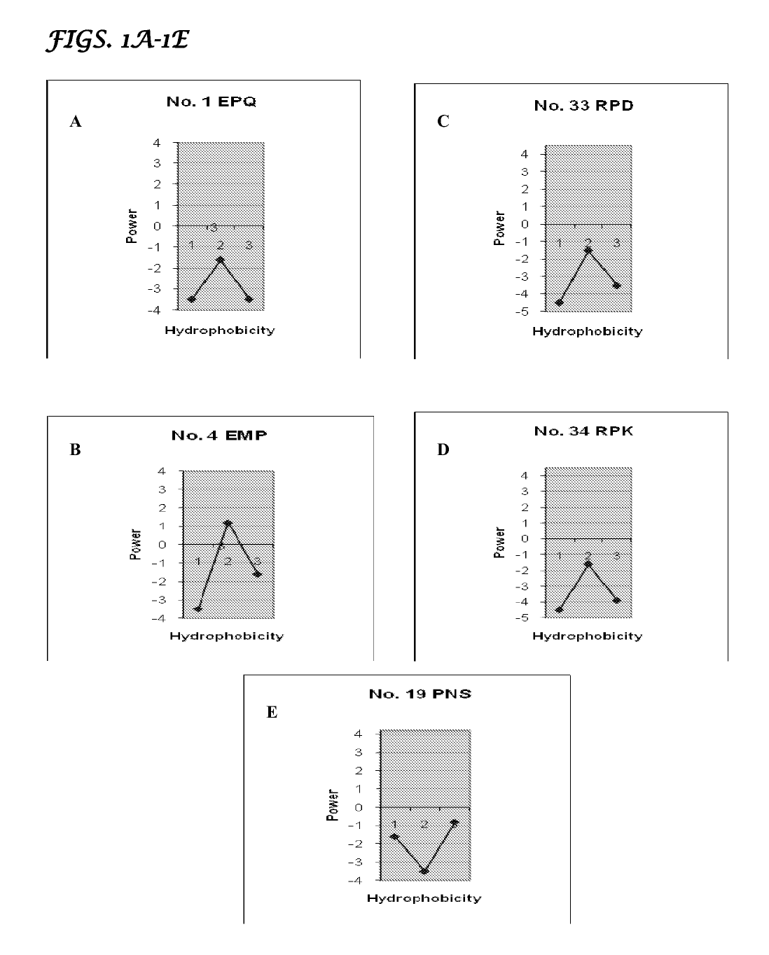

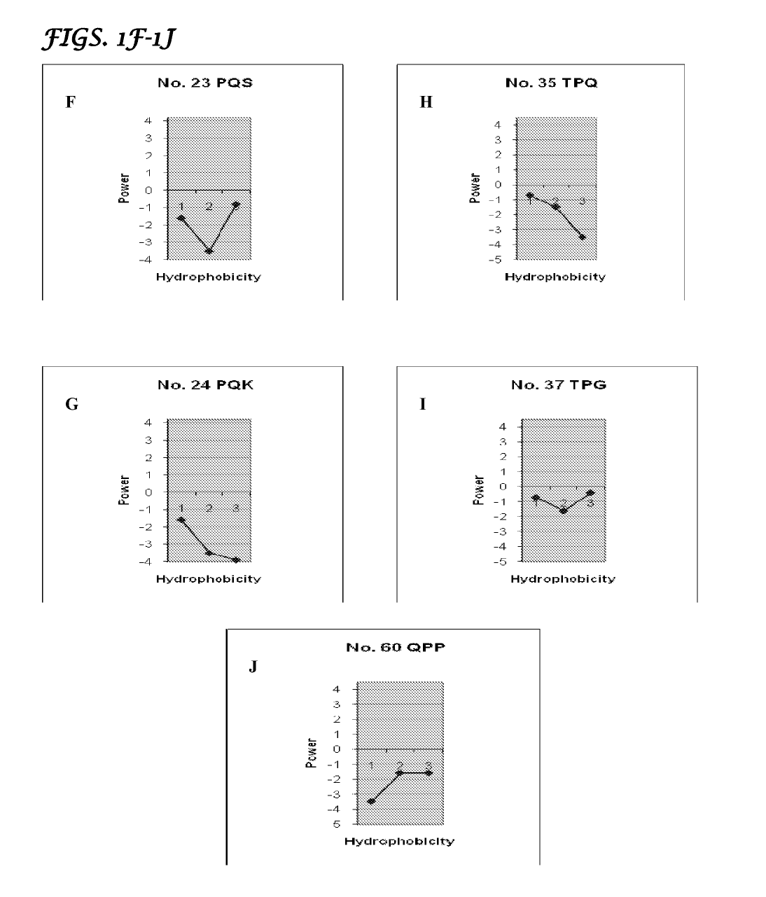

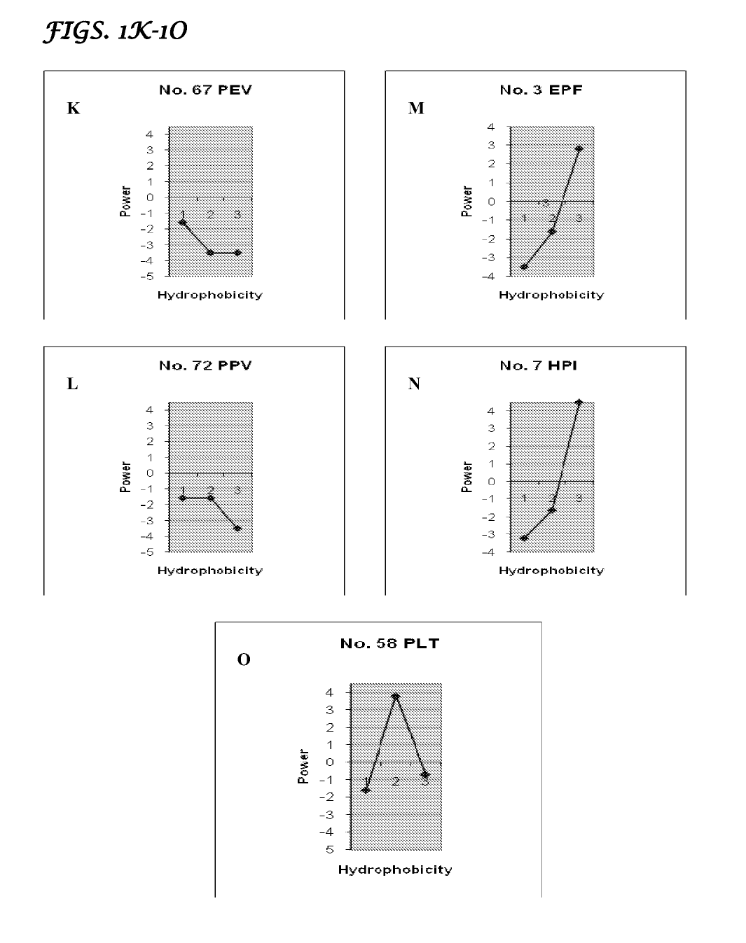

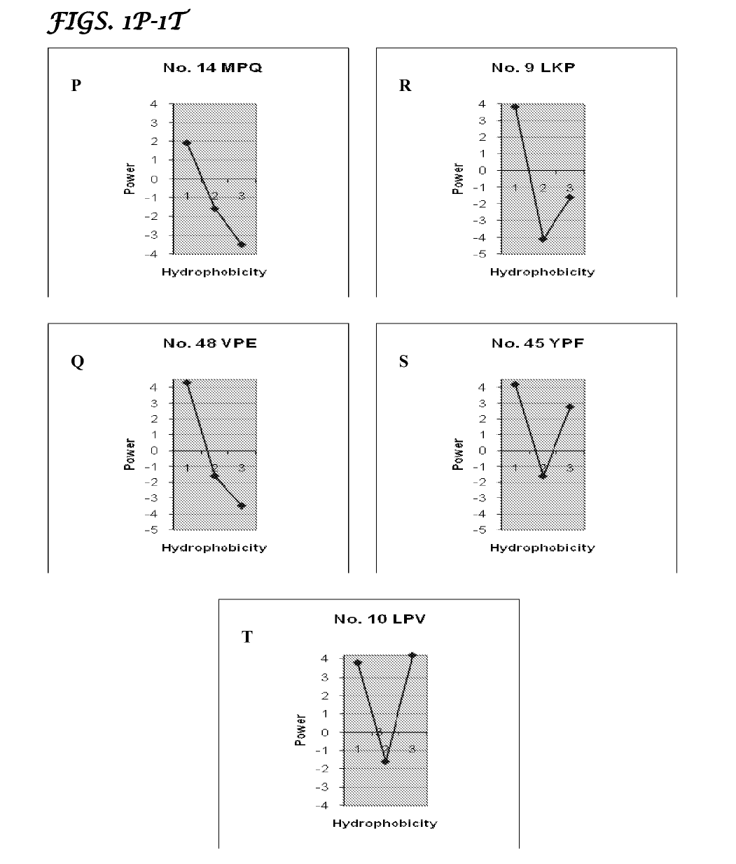

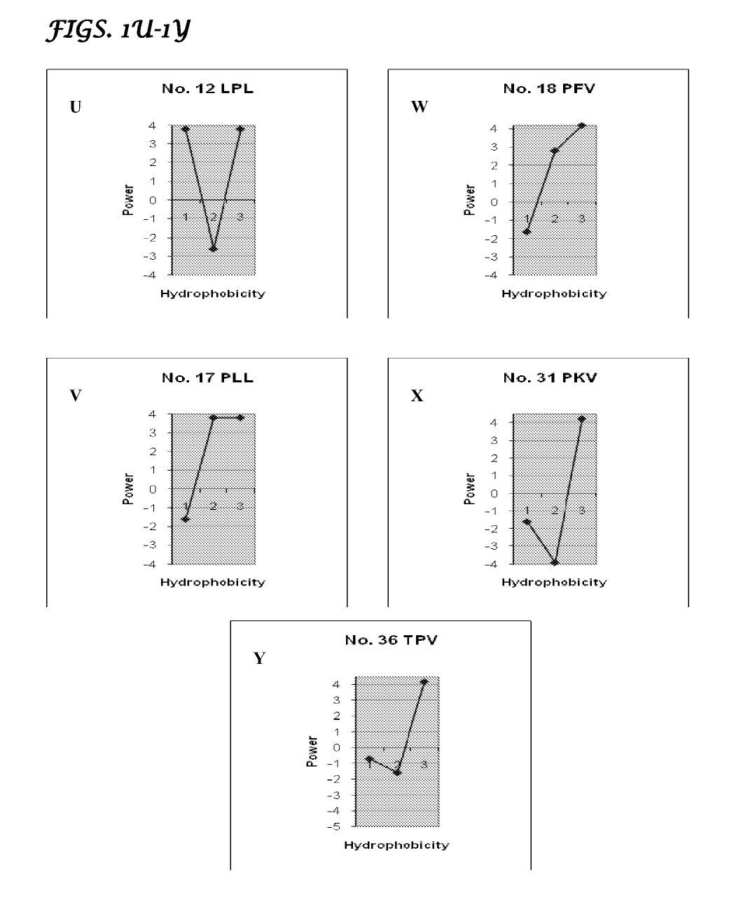

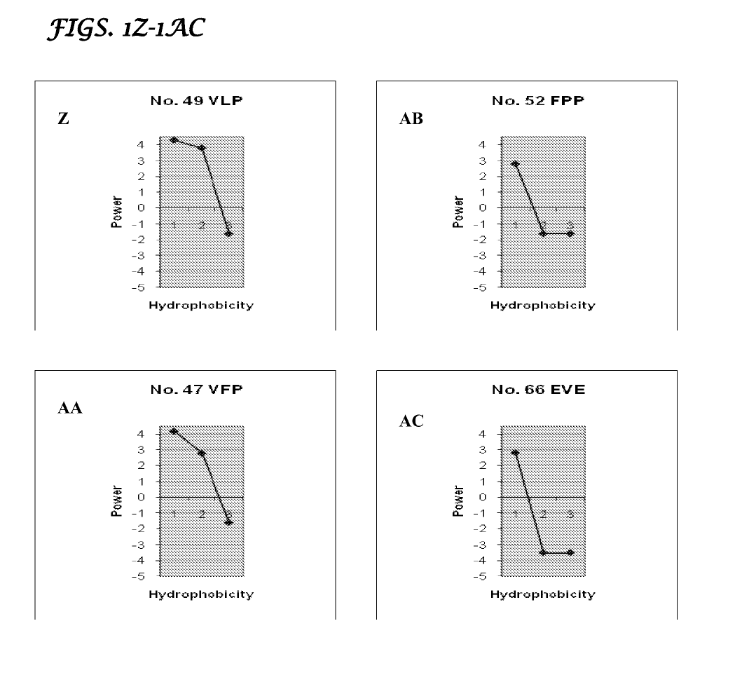

Hydrophobicity analysis of coded in DNA triplets revealed that several show similar hydrophobicity profile as Lisinopril and has similar anti hypertension properties (FIGS. 1A-1AC). Application several similarly acting factors cover broader affected area and elimination mutation mechanism, which may affect the enzyme complex, pressed to escape the controlling attempt. The triplet concentration in body fluids may be also important for diagnostic (modulation) purposes. The diagnostic tool (assay), permitting establish dependency and other variables connected with, will permit precise estimation which element of the body (or metabolism) is affecting the system. The triplets of both networks can be separated according to their hydrophobicity profile into two sub-groups: hydrophilic and mixed. They can be further subdivides according to secondary properties. The examples of hydrophilic and mixed triplets are shown in FIGS. 1A and 1B. Some triplets, within the same sub group may share identical profile even though the triplet was formed from different amino acids. Other combination of the amino acids in the triplets will form different hydrophobicity pattern. Therefore both subgroups of triplet were further subdivided according to the profile. Such classification is shown in FIGS. 1C-1L.

The members of subgroups can be further subdivided in to having very strong, strong and weak hydrophilic profile. Further distinction that should be taken in to consideration is based upon: descending, ascending, broken up, down or flat profile. The subgroups of “mixed triplets” are shown in FIGS. 1M-1AC.

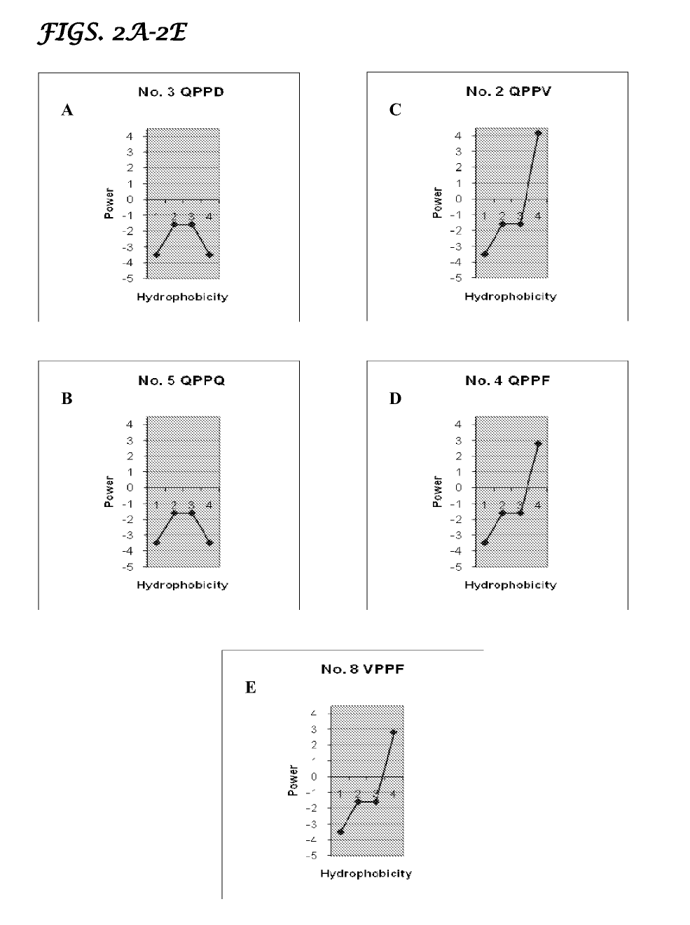

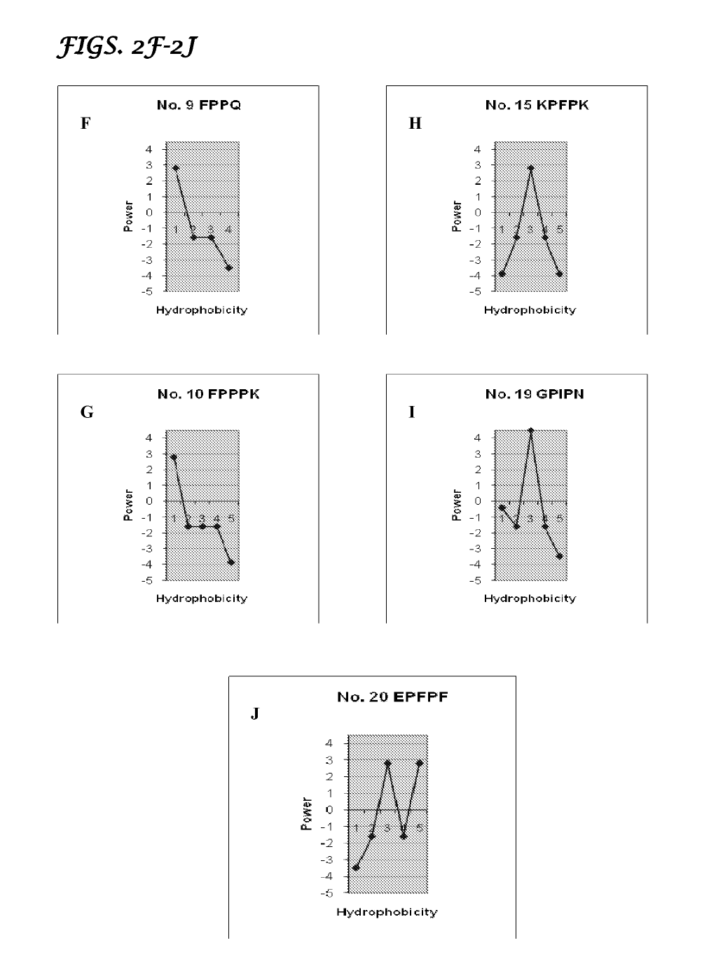

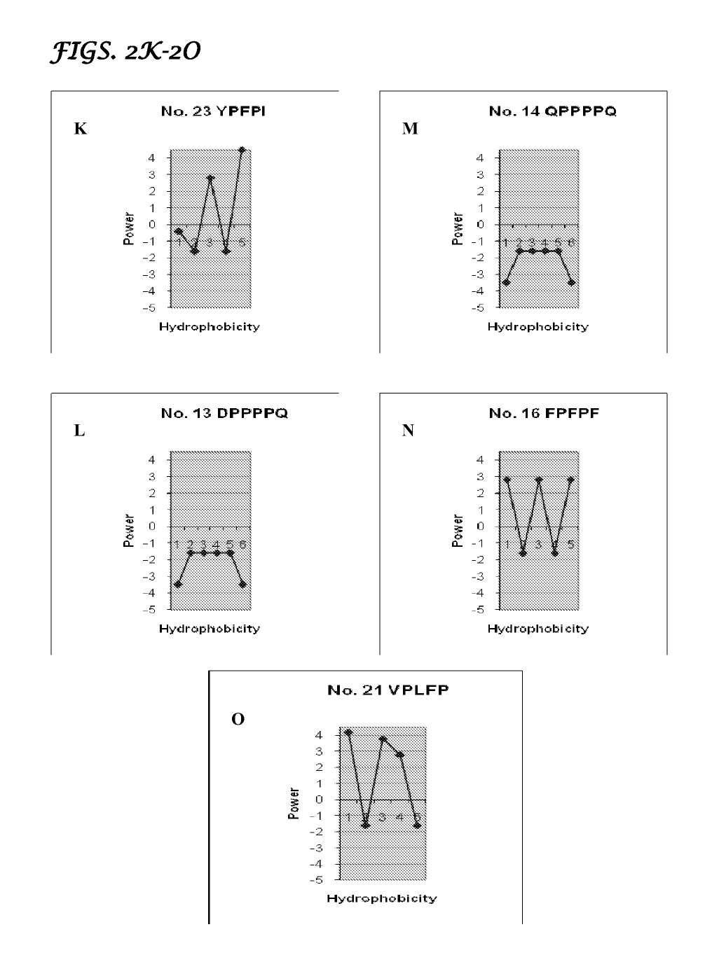

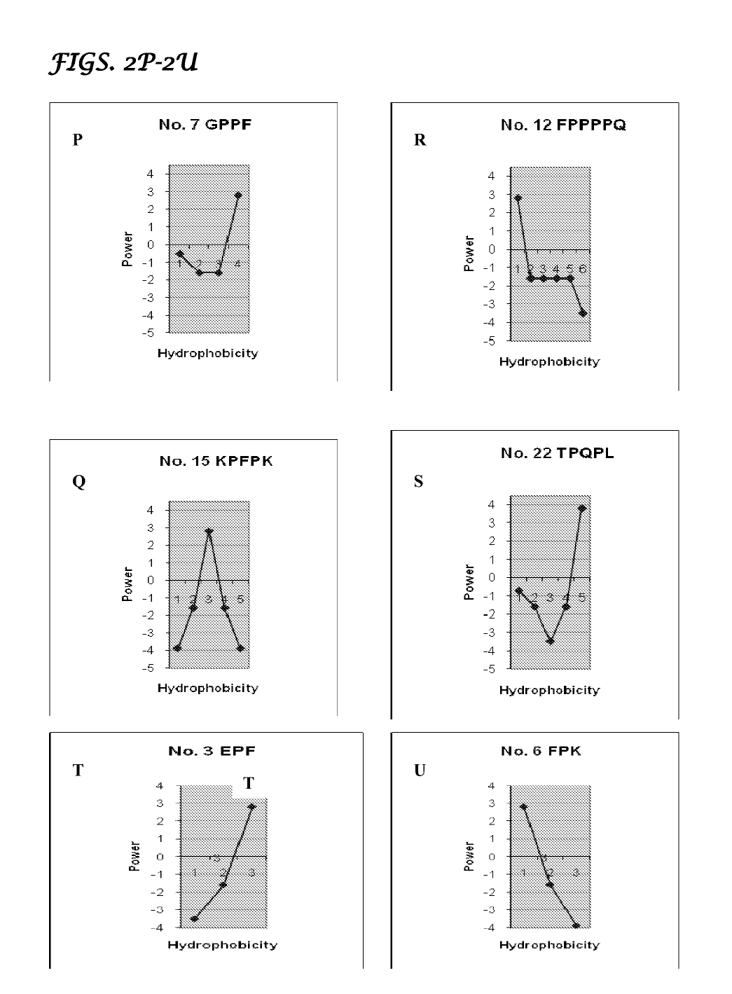

Complex triplets were analyzed for their hydrophobicity profile in similar fashion as simple triplets. The hydrophobicity profiles are shown in FIGS. 2A-2U.

Ovine, Caprine, Bovine, Equine and Human colostrums were collected. Ovine colostrums served as positive control. Bovine Colostrums was isolated from two breeds of Cows, black-white and red-white. Samples were collected within 24 after calving and supplied by the University of Environmental and Life Sciences, Wroclaw, Poland. The Sheep and Caprine colostrums were collected within 24 h after parturition. They were purchased from local farmers. Human colostrums donated by a healthy mother were collected over 62 h starting at 18-h post partum. The PRPC was purified from frozen material using methanol as extracting agent and evaporation of alcohol and water was achieved in vacuum, as previously described in the literature.

The yield of extract was measured and molecular composition was established by chromatography in poly acryl amide gel according to Schägger and von Jagow method (1987). Protein content was determined spectrophotometrically at 280 nm. SDS-PAGE analysis was carried out under reducing conditions. The ultra-low-range molecular-weight marker (ULR MWM) calibration kit was used as a reference. Staining was done with 1% Coomassie Brilliant Blue G 250 (Sigma-Aldrich Co. St. Louis, Mo., U.S.A.) The amount of material isolated from Ovine, Caprine, Bovine and Human colostrums were comparable. The yield from each batch of colostrums was circa 1 mg per 10 ml of the starting material.

SDS-PAGE analysis by Sokolowska et. al.² of different PRPC preparations purified according to Kruzel et al.³ from Ovine, Bovine, Goat and Human colostrums respectively indicates that the electrophoresis pattern of PRPC isolated from Ovine, Bovine, Caprine and Human species look similar but is not identical. The differences may be due to the time of colostrums harvest or the individual nature of isolated peptides species. The collected data shown in chromatogram picture indicates that some fractions present in Ovine colostrums, are seen also in material prepared from Bovine, Caprine and Homo sapiens species.

The samples were collected immediately after the lamming, than the samples were collected after 24, 28, 72, 96, and 120 hrs. The PRPC was isolated by methanol extraction method and tested for individual peptides by ELISA method, using mono-specific antibodies. Within a week of testing, the composition of peptides changed significantly. The findings support the hypothesis that they carry information to cellular computers.

The inventors present a simple, fast, and economical method for the preparation of monospecific antibodies to triplets and amino acids that can be utilized in the following areas: (i) determination of concentration of triplet in body fluids during the life spun, (ii) determining level of informers in different pathological situations, (iii) establishing the role of triplets in chronic diseases, and (iv) designing peptides combination for particular chronic disease. Using this newly developed method of the present invention it is possibly to identify changes within single triplet in PRPC complex.

The basic problem in preparation antibody for triplets and PRPC peptides is the size of antigen use for immunization purposes. Triplets by definition are made from three amino acids. Under normal condition such peptides are too small to induce the antibody production. The monospecific antibodies, however, can be made by presenting the antigen in the form of a single amino acid as an annealed antigen. The annealed antigen can be made from the same amino acid. In other approach it is possible to increase the size of antigen by repeating the same motif several times. Formation of the annealing polymer will overcome the difficulties connected with the antigen (peptide) for antibody production.

In order to make annealing it is necessary sometimes, to add to peptide empty (not active immunological) amino acids. The strategy used for production antibodies included both variants and addition of the complete Freund adjuvant. The mono-specific antibodies against various ovine PRPC antigens were prepared on rabbits. The larger animals like sheep, goat, or horse may also be utilized. Two rabbits were selected for each natural antigen. The designed procedure resulted in production of very strong antibodies shown in the Table 1.

TABLE 1
The estimated the titer of the antibodies, in sera from the
immunized rabbits is shown below.

| Peptide | Before immunization | Post immunization titers. |
|---|---|---|
| SEQ. ID. NO.: 1 | 0 | 12,800 |
| SEQ. ID. NO.: 2 | 0 | >25,600 |
| SEQ. ID. NO.: 3 | 0 | 12,800 |
| SEQ. ID. NO.: 4 | 0 | >25,600 |
| SEQ. ID. NO.: 5 | 0 | >25,600 |
| SEQ. ID. NO.: 6 | 0 | 6,400 |
| SEQ. ID. NO.: 11 | 0 | >25,000 |
| SEQ. ID. NO.: 12 | 0 | >25,000 |
| SEQ. ID. NO.: 13 | 0 | >25,000 |
| SEQ. ID. NO.: 14 | 0 | >25,000 |
| SEQ. ID. NO.: 15 | 0 | >25,000 |
| SEQ. ID. NO.: 16 | 0 | >25,000 |
| SEQ. ID. NO.: 17 | 0 | >25,000 |
| SEQ. ID. NO.: 18 | 0 | >25,000 |
| SEQ. ID. NO.: 19 | 0 | >25,000 |
| SEQ. ID. NO.: 20 | 0 | >25,000 |
| SEQ. ID. NO.: 21 | 0 | >25,000 |
| SEQ. ID. NO.: 40 | 0 | >25,000 |
| 1 Lacto 1 | 0 | >25,000. |

The antibodies were tested for their specificity by ELISA method against 20 different synthetic peptides selected from the sub-groups A, B, D and other. Results are summarized in Table 2.

Information presented in Table 2 indicates that several antigens induced specific antibodies. In majority cases however, cross reaction ware present even it was rather weak, and easy to eliminate i.e. by increase dilution of the antibody or absorption of non specific antibody on immune columns. The antibody to SEQ. ID. NO.: 12 peptides reacted in specific manner. Three other antibodies SEQ. ID. NOS.: 52, 17 and 40 are reacting with other antigen, although the cross reactions was relatively weak. Simple dilution of antibodies could eliminate non specific reaction.

Antibody: SEQ. ID. NOS.: 11, 20, and 3 reacted rather strong with heterogonous antigens. In native form they were useless but could be rescue after adsorption non specific antigens and the former use as monospecific antibody detecting specific epitopes of the triplet. Also the antibodies to SEQ. ID. NOS.: 13, 17, and 21 antigens cross reacted with different antibodies in non specific manner can be re-specified by proper adsorption. They can be still rescue and use after elimination non specific antibodies.

TABLE 2
Cross-reactive antibodies present in mono-specific anti PRPC sera.

| An- | | | | | | | | | | | | | | | | | | | | |
|---|---|---|---|---|---|---|---|---|---|---|---|---|---|---|---|---|---|---|---|---|
| tigen, | | | | | | | | | | | | | | | | | | | | |
| A- | | | | | | | | | | | | | | | | | | | | |
| body | 40 | 20 | 21 | 18 | 11 | 17 | 16 | 15 | 12 | 14 | 13 | 12 | 3 | 4 | 1 | 2 | 5 | 7 | Lact. | 9 |
| 1 | 0 | 0 | 200 | 0 | 0 | 0 | 0 | 0 | 0 | 0 | 400 | 0 | 0 | 200 | 1600 | 0 | 0 | 0 | 0 | 0 |
| 19 | 0 | 0 | 400 | 0 | 0 | 0 | 0 | 0 | 0 | 0 | 400 | 0 | 0 | 400 | 0 | 0 | 0 | 0 | 0 | 1600 |
| 13 | 0 | 0 | 400 | 0 | 200 | 200 | 0 | 0 | 0 | 800 | 1600 | 0 | 0 | 200 | 0 | 0 | 0 | 0 | 0 | 0 |
| 2 | 0 | 0 | 0 | 0 | 800 | 400 | 0 | 0 | 0 | 0 | 1600 | 0 | 0 | 200 | 0 | 1600 | 0 | 0 | 200 | 0 |
| 18 | 0 | 0 | 0 | 1600 | 0 | 0 | 0 | 0 | 0 | 0 | 1600 | 0 | 0 | 0 | 0 | 0 | 0 | 0 | 0 | 0 |
| 5 | 200 | 0 | 200 | 0 | 0 | 0 | 0 | 0 | 0 | 0 | 200 | 0 | 0 | 0 | 0 | 0 | 800 | 0 | 0 | 0 |
| 7 | 0 | 0 | 0 | 0 | 0 | 0 | 0 | 0 | 0 | 0 | 400 | 0 | 0 | 0 | 0 | 0 | 0 | 1600 | 0 | 0 |
| Lacto | 0 | 0 | 400 | 0 | 0 | 0 | 0 | 0 | 0 | 0 | 800 | 0 | 0 | 0 | 0 | 0 | 0 | 0 | 1600 | 0 |
| 21 | 0 | 0 | 0 | 0 | 0 | 200 | 0 | 0 | 1600 | 0 | 800 | 0 | 0 | 0 | 0 | 400 | 0 | 0 | 0 | 0 |
| 12 | 0 | 0 | 0 | 0 | 0 | 0 | 0 | 0 | 0 | 0 | 0 | 1600 | 0 | 0 | 0 | 0 | 0 | 0 | 0 | 0 |
| 40 | 1600 | 0 | 0 | 0 | 0 | 0 | 0 | 0 | 0 | 0 | 400 | 0 | 0 | 0 | 0 | 0 | 0 | 0 | 0 | 0 |
| 14 | 0 | 0 | 0 | 0 | 0 | 400 | 0 | 0 | 0 | 1600 | 400 | 0 | 0 | 0 | 0 | 0 | 0 | 0 | 0 | 0 |
| 20 | 0 | 1600 | 800 | 400 | 0 | 1600 | 0 | 0 | 200 | 400 | 1600 | 0 | 0 | 800 | 200 | 0 | 0 | 0 | 400 | 400 |
| 3 | 0 | 0 | 0 | 0 | 1600 | 0 | 1600 | 200 | 0 | 400 | 800 | 0 | 1600 | 200 | 0 | 0 | 0 | 0 | 0 | 0 |
| 4 | 0 | 0 | 0 | 200 | 1600 | 0 | 0 | 0 | 0 | 0 | 0 | 200 | 0 | 1600 | 200 | 0 | 0 | 0 | 0 | 0 |
| 21 | 0 | 0 | 1600 | 0 | 0 | 0 | 0 | 0 | 200 | 0 | 200 | 0 | 0 | 0 | 0 | 0 | 0 | 200 | 400 | 0 |
| 11 | 0 | 0 | 400 | 0 | 1600 | 1600 | 0 | 0 | 0 | 400 | 200 | 200 | 0 | 0 | 400 | 0 | 0 | 0 | 0 | 0 |
| 17 | 0 | 0 | 0 | 0 | 0 | 1600 | 0 | 0 | 0 | 0 | 800 | 0 | 0 | 200 | 0 | 0 | 0 | 0 | 0 | 0 |
| 16 | 0 | 0 | 0 | 0 | 0 | 400 | 1600 | 0 | 0 | 0 | 800 | 0 | 0 | 0 | 0 | 0 | 0 | 0 | 0 | 0 |
| 15 | 0 | 0 | 0 | 0 | 0 | 800 | 0 | 1600 | 0 | 0 | 0 | 0 | 0 | 0 | 0 | 0 | 0 | 0 | 0 | 0 |

The instant invention provides evidence that the living organism protects and utilizes information stored in informer peptides (the Alphabet PRPC). The informer-peptide carries species specific information provided by mother and the stem cells. Originally stem cells are present in the mother's mammary gland and delivered to offspring with colostrums and milk (as mentioned earlier). The information was selected by ancestors for the offspring, to facilitate their early life. The human and animals species keep that information to feed the CN and repair mechanism of the host. After weaning, during the rest of the life spun, CN III, CN IV and CN V receive peptides from the stem cells.

Before their use the information is stored in type II storage facilities located on the macromolecules. Each species of macromolecules have hydrophilic and hydrophobic regions like i.e. gamma globulins. The triplets, highly charged attach to macromolecule surface and remain protected until the time when they find right place where they are needed. The system fills the time gap between emergency demand and synthesis new generation of peptides-informers. Programming new peptides depends upon information delivered by the complex. The computer programming the new cells, which replace used or dead cells, represents a continuous process occurring in the body from the birth to the host death. This newly discovered mechanism represents key phenomenon that permits the host to program population of short life spun cells as well as cells operating in gland and organs including the host communication.

The small PRPs described herein carries information collected by ancestors, to facilitate the offspring life. Peptides are carrying information in form of simple commands or programs, addressed to the host organs through the communication networks. The host has to its disposition five different communication networks. One of the communication networks called old or CN IV, was formed before DNA/RNA system was adapted to the cells. This group of informers-triplets-chaperones modulates cellular metabolism, providing to cells information how to protect the cell from oxidative stress or environmental insults and how to repair induced damages. These classes of triplet are multiply using old fashion alternative method of protein synthesis which today is still in use.

The second group of triplets called Communication network III (CN III) consist of peptides and triplets coded on DNA. They serve host as chaperones, controlling processes occurring in organs or whole body. They play significant role in defend the host against development chronic infectious diseases like: Tuberculosis, Leprous, Hepatitis type B and C, Herpes infections. The triplets located in CN III play also significant role in modulating Cytokine-Hormones networks (CN II) function. In addition peptides of CN III group have capabilities to inhibit mutation processes by providing information how to prevent mutations.

Both carriers (CN IV and CN III) operate as species specific entity having characteristic hydrophilic profile. The model described herein hypothesizes, constant flow of information to new cells, substituting decayed cell population. The information is provided in early phase by colostrums and milk and after the weaning by Stem cells. To achieve the goal, part of information is stored in facility type II for this to be true; we need to find storage facilities, beside the DNA, for not coded on DNA chaperones-informers. It was postulated that storage system type 2 may be on the surface of a different macromolecule (s) circulating in the body fluids and amino acid proline is capable of storing information thanks to cis-trans-cis configuration. Stabilization of stored information is served by satellite amino acids with a particular hydrophobicity property.

The discovered mechanism of protection and transportation of peptides and triplets that are biologically and chemically very active molecules opens several possibilities for modulation metabolic processes till now not known. Formulated in the body fluids macromolecules have negatively and positively charged regions to which the triplets from both networks can attach and stay protected until the host needed them. The need for release triplets is manifested achieved by changes in pH, salt concentration, elevation the temperature or presence of free radicals. This change affects the configuration of macromolecule causing release the bind triplets. The storage facility hypothesis was supported by finding different hydrophobic configurations of the proline rich peptides and triplets. The differences are summarized in Table 3.

TABLE 3
Hydrophobic index of the proline rich peptides present
in different sub-groups of ovine PRPC.

| | | Hydrophobic | Individual |
|---|---|---|---|
| | No. of Peptides | index | Charge |
| Sub-group | In group | in group | (Average) |
| A | 14 | −48.3 | −3.45 |
| B | 17 | +36.1 | +2.12 |
| C | 36 | −70.8 | −1.97 |
| D | 1 | −19.1 | −19.1 |
| E | 14 | −11.7 | −0.84 |

Presented information concerning hydrophobicity of PRPC clearly support discovery presented previously. Analysis the hydrophobicity profiles permitted to postulate that each sub-group can carry different information and this information in connected with specific triplets. The specific group of peptides can be released back in to the body fluid by changing described above environmental condition. Normally triplets are protected against non-specific binding by peptides-transporters with hydrophobicity or hydrophilic regions to which the triplets are adsorbed.

The ovine PRPC purified as described earlier was applied on immobilized IgG-2 column at neutral pH and eluted in pH 5.0 buffer. The starting materials not adsorbed and eluted materials were tested for the presence of selected peptides by an ELISA method, using mono-specific anti peptide antibody. The results of the studies are shown in Table 4.

TABLE 4
An evaluation the Proline Rich Peptides on immobilized
gamma globulin column.

| | Peptide | | | | | | |
|---|---|---|---|---|---|---|---|
| | A-1 | A-3 | B-8 | B-9 | C-2 | C-11 | D-1 |
| Titer in pool | 5400 | 1350 | 9000 | 2075 | 5325 | 15400 | 9300 |
| Not adsorbed | 800 | 400 | 400 | 0 | 0 | 400 | 200 |
| Desorbs | 3200 | 6400 | 12800 | >25600 | 12800 | >25600 | 6400 |
| (pH 5.0) | | | | | | | |

The data presented in Table 4 demonstrates differential binding of tested peptides. Changing the pH 7.0 of the IgG column by the elution buffer of pH 5.0 releases the peptides from the immobilized gamma globulin. Since the interaction between gamma globulin and peptide is not specific, the adsorption and desorption of the peptides from PRPC confirm theory concerning storage facility No II. Furthermore, the finding supports the hypothesis that binding the triplets to macromolecule represents the new mechanism used by host to transport triplets to new cells.

The second group of chaperon-informers made by DNA can be stored in the beta casein, as it happens in colostrums and milk. Beta casein coded peptides provide the offspring with essential part of information during the feeding period. Lack of material in baby food may cause in the offspring serious sequels connected with the failure of programming the cellular computer.

The development serious sequels like premature aging, dementia or death of the progeny. Discovered mechanism of transmission the information represents a new phenomenon present not only within humans but also in other species of mammals.

The impacts may not be necessary identical in different species. They may be coded differently, and looks different. For that reason they are understood only by species producing information. Only exception represent those triplets that were produced at the time, when forebears were still lived in the common pool, and was made by undifferentiated cells. At such early stage, the ancestors had not developed operational DNA/RNA system. The primitive organisms were utilizing alternative protein synthesis, a system use today by prions. For today's host, the old fashion mechanism has still several advantages: The information present on PRPC and triplets has to be delivered to CN fast. This is particularly important in an emergency situation. This problem is partially covered by Storage system II but it does not answer how the host can expeditiously replace the exhausted pool of informers. How much time, such process last, can be estimated by following the responses to vaccination. The induction specific antibodies to new antigen require circa 7-12 days. During this period, the invading organism can overcome innate defenses and kill the host. The invading pathogens divide with the speed of every 5 minutes one generation, leading to appearance 288 generations within 24 hrs. As calculations indicate, the host in the emergency situation, has no time for waiting for needed information stored somewhere in the DNA library. The emergency situations may occur any minute of the host life. For example, in the gastro-intestinal tract are thriving circa 4 lbs of pure germs. They are ready to attack the host any time when innate barrier fail. During the host life span, the germs are limited to the lumen of the intestinal tract, because of the innate barrier. However, fifteen minutes after the host death the germs present in intestinal tract cross the intestinal track barrier and spread thought the body. There are other barriers inside the host. These facts illustrates how powerful the innate defense system is and what consequences may occur when the system is damaged. Presented example illustrates how suddenly the crisis may arise.

To keep control over such large number of potentially invading saprophytic microorganisms and toxic elements from an environment, whole army of cells-defenders, and numerous non specific barriers were installed in host. Effective cooperation between short life span cells and humoral defenses and by chaperon-informers system present in CN III, and CN IV effectively protect the host. The process of programming require delivery to new cells proline rich peptides and in particular the triplets. The triplets are particularly important because they do not require special activation. Adsorption to macromolecules looks like routine process, which permits the peptides to take over the responsibility from the mother to initiate programming the new cells.

Recent studies revealed that in each organ mentioned above the new cell are born and they ought to replace the one who died. The new cells before taking responsibility had to be program to prepare for future activity. The efficacy of adaptation depends upon arrival informing the molecules with the programs. The absence of triplets needed, for biological computer inevitable lead to, the premature death of the new cells. This rule holds for different type of cells operating inside the body. With the aging the programming process deteriorates more rapidly and cells originally present in secretory gland organs and brain start to die out. At certain moment(s) generated cells present in the organs die more rapidly than the new cells are formed to (can) replace them, leading to what may be called as beginning of chronic progressive degenerative disorder, not reversible condition and always leading to failure of the host. The aging or AD may serve as an example of chronic progressive deterioration of the brain function. Presented mechanism of development chronic progressive deterioration of the host function can be seen not only in humans but also in all other mammals. In summary the instant invention describes a novel discovery of the triplet multiplication methods and modulation in the body through the circulating in body fluids, the storage system, and the new mechanisms allowing safe transportation PRPC and triplets to the recipient.

Food deficiency of new type occurs as result of the inadequate delivery of the nutrition elements. Inadequate supply of amino acids and proline rich peptides lead to development systemic malfunctions in the organs and cells, leading to chronic progressive diseases that shorten the host life spun. New types of the food deficiencies occur most frequently among offspring fed during early phase of the infancy with nutrients substituting mother's products. Such infants fail to develop correctly and exercise problems with transfer from embryo to mature individual. The elements needed for transformation, from embryo state, to early childhood are present exclusively in mother colostrums and milk. Since information provided by mother, during this period of time are species specific applications substituted food by baby formulas disturb normal and correct development of the progeny. Substitution by baby food formula results in a lack of key information for correct development and programming of cellular biological computers further leading to malnutrition. The sign are manifested by appearance of various disorders affecting the progeny life.

The new type of food deficiency may occur as result of the depletion three essential elements present in mother's colostrums and milk.

The first represent group of food deficiencies occurring due to not adequate supply of certain amino acids. Since the newborn can't synthesize all amino acids supplementation wrong amino acid concentrations may lead to different types of malfunction like allergy. Development allergy to food (i.e. milk) will force to apply anti allergic food supplements, which further distance food from physiological needs of the new born. The amino acids; Ala, Leu, Pro and Val are in much higher concentration in mother breast's products than they are present in the Baby food formulas. Balancing lack of amino acids by old fashion way (addition more proteins to the food will further disturb the protein balance).

An example of possible errors are shown in Table 5 where concentrations of amino acids present in Proline Rich Peptide Complex (PRPC) isolated from ovine colostrums are listed and compared with values present in different protein.

Table 5 presents information collected from different series of colostrinin preparation. As may be seen the concentration of the amino acids are too high or too low to norms for adult protein. They are in PRPC statistically different concentrations, than in the adult protein. At the same time concentration of the Asp, Arg, and Ile ware below concentration present among adult proteins. The other differences between preparations are present but are not statistically significant. Application such unbalanced food lead to development different types of intolerances. Perhaps the most important defect is induced in programming the cellular biological computer. Substitution the mother's milk by products from adult will induce statistically significant depletion or oversupply the amino acid that the offspring can't handle at this stage of life because its own production of amino acids is not ready to correct or compensate the existing differences. In addition supply coded material (PRPC) in food prepared from heterospecies sources dipped the food deficiency further. This further alters the process of cellular biological computer programming. Table 6 presents the amino acid composition of PRPC isolated from colostrums of different species.

TABLE 5
The amino acid compositions of five batches of the ovine PRP
preparations are expressed in %.

| | | | | | | | | Avg. |
|---|---|---|---|---|---|---|---|---|
| | | | | | | | | Amino |
| | | | | | | | | Acid |
| | | | | | | | | from |
| Amino | | | | | | | | 200 |
| Acid | PRP1 | PRP2 | PRP3 | PRP4 | PRP5 | Avg. | Control | proteins |
| Asp/Asn | 2.14 | 2.92 | 2.63 | 3.12 | 2.56 | 2.7 | 5.5 | 0.49 |
| Ser | 5.36 | 5.54 | 5.6 | 5.26 | 5.27 | 5.44 | 7.1 | |
| Glu/Gln | 13.7 | 15 | 15.39 | 15.13 | 14.9 | 14.8 | 3.9 | 3.8 |
| Gly | 3.04 | 3.3 | 3.32 | 2.77 | 2.32 | 3.11 | 2.5 | |
| Hist | 2.92 | 2.15 | 2.45 | 2.39 | 1.94 | 2.48 | 2.1 | |
| Arg | 3.26 | 1.85 | 1.83 | 3.11 | 1.8 | 2.5 | 6.02 | 0.41 |
| Thr | 6.02 | 4.97 | 5.13 | 6.99 | 6.55 | 5.78 | 6 | |
| Ala | 4.42 | 3.32 | 3.38 | 2.62 | 1.38 | 2.94 | 9 | 3 |
| Pro | 20.42 | 21.97 | 20.58 | 21.5 | 22.9 | 21.12 | 4.6 | 4.6 |
| Tyr | 1.61 | 1.54 | 1.41 | 1.29 | 1.62 | 1.46 | 3.5 | 1.7 |
| Val | 14 | 10.78 | 10.79 | 11.87 | 12.85 | 11.15 | 6.9 | 1.6 |
| Met | 3.24 | 3.53 | ND | 3.93 | 3.39 | 1.7 | 1.99 | |
| Lys | 5.93 | 4.79 | 5.8 | 5.5 | 7.16 | 5.5 | 7.0 | |
| Ile | 2.48 | 2.74 | 2.85 | 3.05 | 2.48 | 2.87 | 4.6 | 0.63 |
| Leu | 12.1 | 11.5 | 11.13 | 11.12 | 9.6 | 11.47 | 7.5 | 1.53 |
| Phe | 5.08 | 4.37 | 4.2 | 4.31 | 4.72 | 4.49 | 3.5 | |

TABLE 6
The Amino acid composition of PRPC isolated from
colostrums of different species.

| Amino acid | Ovine | Bovine | Caprine | Human | Control* | Index | Hu/other |
|---|---|---|---|---|---|---|---|
| Asp/Asn | 2.80 | 4.89 | 3.21 | 6.26 | 5.99 | 1.04 | 0.58 |
| Ser | 4.05 | 6.42 | 5.71 | 2.46 | 7.10 | 0.35 | 2.19 |
| Gln | 15.77 | 14.98 | 16.93 | 15.82 | 10.10 | 1.58 | |
| Gly | 3.03 | 3.45 | 2.84 | 1.64 | 7.50 | 0.21 | 1.90 |
| Hist. | 2.14 | 2.20 | 2.24 | 2.37 | 2.10 | 1.12 | |
| Arg. | 3.34 | 2.20 | 1.90 | 1.65 | 4.70 | 0.35 | |
| Thr | 5.30 | 5.07 | 4.90 | 3.83 | 6.00 | 0.63 | |
| Ala | 2.13 | 3.48 | 1.36 | 4.80 | 9.00 | 0.54 | 0.48 |
| Pro | 22.50 | 21.32 | 20.50 | 22.14 | 4.60 | 4.81 | |
| Tyr | 1.54 | 2.00 | 1.91 | 2.42 | 3.50 | 0.69 | |
| Val | 11.50 | 9.36 | 10.44 | 9.70 | 6.90 | 1.41 | |
| Met | 1.70 | 1.79 | 3.22 | 1.47 | 1.70 | 0.86 | |
| Lys | 4.93 | 5.41 | 4.53 | 4.08 | 7.00 | 0.58 | |
| Ile | 3.42 | 4.39 | 3.81 | 6.01 | 4.60 | 1.30 | 0.64 |
| Leu | 10.47 | 8.63 | 11.53 | 12.82 | 7.50 | 1.71 | |
| Phe | 4.77 | 4.42 | 4.97 | 2.28 | 3.50 | 0.65 | 2.07 |

The information presented in Table 6 reveals, that amino acids present in different PRPC exhibit different amino acid concentration pattern when compared with control. That pattern reflects different set of triplets participating in biological computer programming. The concentration amino acids within species are comparable but they differ from average values of amino acids seen in adult proteins. This is the proof that heterospecies colostrums and milk can't substitute mother breast products.

The differences between the species however are not so large, so the substitution PRPC from other sources is possible, but heterospecies PRPC can't provide information coded in homospecies PRPC. This facts, needs to be taken in to consideration. The triplet's composition in different species is different. Specificity of informers-chaperones units present in different species of PRPC is different. The triplets, in every tested species, are formulated from the same amino acids, but they are design in different fashion so they can't be utilized by heterospecies organism. In this situation, colostrums and milk from other species can be use only as a source of amino acids, scavengers and energy. Thus, application protein from other species or sources is not advisable. Additional problem creates fact that colostrums and milk have higher concentration of certain amino acids then they are in adult proteins, e.g., Proline. The reverse situation is with amino acid Serine. The highest concentration was found in bovine species and the lowest in Human.

Based on presented data it can be concluded that substitution the food for the offspring by proteins from other sources than mother does not meet newborn requirements. To address, whether the similar pattern of proline rich peptides, are observed among isolates from different colostrums form similar or different amino acid pattern the inventors performed a study. The beta caseins sequences from different species were obtained from Protein data Bank. They were aligned and composition of amino acid in triplets present in beta caseins was calculated for different species. Results are presented in Table 7.

Data presented in Table 7 revealed, that amino acid concentration in peptides coded in Beta casein practically exhibits similar pattern than amino acids isolated from colostrums. These studies revealed that correction the amino acids composition present in baby food, by addition the animal or plant protein is simply not advisable. The amino acids from animal or plant sources can serve only as substitution for amino acid or energy. All informatory material has to be delivering by mother or adult Stem cells to achieve desired affect. Lack of information or programs from natural sources may cause serious metabolic disturbances in recipient function.

TABLE 7
Amino acid compositions present in triplets of
different species of Beta caseins.

| Amino | | | | | | |
|---|---|---|---|---|---|---|
| acid: | Ovine | Bovine | Equine | Human | Control | C/H index. |
| A. Ala | 2.69 | 4.48 | 5.00 | 4.00 | 2.13 | 0.53 |
| B. Asp | 0.00 | 0.00 | 0.00 | 0.00 | 5.50 | 0.00 |
| C. Cys | 0.45 | 0.45 | 0.44 | 0.44 | 0.44 | 1.00 |
| D. Asp | 2.42 | 2.42 | 6.47 | 3.34 | 10.10 | 3.02 |
| F. Phe | 4.04 | 3.32 | 1.78 | 3.50 | 1.97 | — |
| G. Gly | 1.74 | 2.42 | 2.49 | 0.89 | 7.50 | 8.43 |
| H. His | 1.74 | 2.69 | 1.25 | 1.78 | 2.10 | 1.18 |
| I. Ile | 4.04 | 4.48 | 4.56 | 6.23 | 4.60 | 0.74 |
| K. Lys | 5.38 | 5.83 | 4.56 | 1.89 | 7.00 | 3.70 |
| L. Leu | 11.66 | 12.56 | 11.20 | 13.78 | 7.50 | 0.54 |
| M. Met | 1.14 | 1.79 | 2.49 | 1.78 | 1.70 | 0.96 |
| N. Asn | 1.74 | 1.79 | 1.66 | 1.78 | — | — |
| P. Pro | 15.25 | 16.59 | 14.94 | 17.33 | 4.60 | 0.27 |
| Q. Glu | 9.87 | 9.42 | 9.54 | 10.67 | 10.10 | 0.92 |
| S. Ser | 6.28 | 6.73 | 6.64 | 4.00 | 4.05 | 1.01 |
| T. Thr | 4.04 | 3.57 | 2.91 | 3.52 | 6.00 | 1.71 |
| V. Val | 10.31 | 8.97 | 9.13 | 10.22 | 6.90 | 0.68 |
| Y Tyr | 1.35 | 1.79 | 2.07 | 1.78 | 3.50 | 1.97 |

The food deficiencies, described above, can be considered as a new kind of deficiency that has not yet been addressed. Analysis of the existing situation permits to designed strategies leading to elimination malnutrition and appearance potential diseases and social disaster. Strategies to correct presented nutrition deficits. Presented data further confirm that triplets constructed by different species can't substitute triplets provided by mother for programming cellular biological computers because they are constructed with different amino acids.

I. When adequate concentration of the amino acids are not supplied in the baby formulas adding the lacking amino acids is suggested particularly in form of the triplets since they carry ancestors information selected specially for the offspring.

II. Using synthetic triplet's identical to present in mother colostrums should securely supply the offspring with the triplets and small peptides as described herein. Using this strategy it would be possible to correct lack of informer molecules needed by neonates for programming CN and biological computer. Analysis in depth the structure of the beta casein revealed presence of more complicated structures that may carry whole programs. They should also be included in to triplet's pool. Adaptation proposed strategy is important, since heterospecies populations of triplets and PRPC are useless for the newborn because they do not have structures needed by offspring.

III. Question of selection the carrier molecule remains open. This represents the key to successful substitution of Baby food.

IV. The deficiency in mother's Stem cells, present in the colostrums and the milk should be substituted by adequate supply of members of CN II, CN III and CN IV networks. Secure delivery of Interferon alpha, beta and gamma to create in the neonate the Interferon cascade which protects the newborn against invading germs from the environment. The triplets from CN's should be provides as mentioned earlier.

The present invention provides structures of triplets existing in human colostrums and milk (Table 8).

TABLE 8
The list of triplets identified in human species beta casein.

| No | Peptide | No |
|---|---|---|
| 1 | APV | 1 |
| 2 | AQP | 1 |
| 3 | AVP | 1 |
| 4 | DPQ | 1 |
| 5 | LPL* | 2 |
| 6 | LPV* | 2 |
| 7 | LPP | 1 |
| 8 | LPI | 1 |
| 9 | MPV | 1 |
| 10 | NPT | 1 |
| 11 | NPI | 1 |
| 12 | PQP | 3 |
| 13 | PTI | 1 |
| 14 | PFF | 1 |
| 15 | PQQ | 1 |
| 16 | PLL | 1 |
| 17 | PKY | 1 |
| 18 | PFT** | 1 |
| 19 | PIP | 1 |
| 20 | PKV | 1 |
| 21 | QPA | 1 |
| 22 | QPL | 3 |
| 23 | VPF* | 1 |
| 24 | VPK* | 1 |
| 25 | VPQ | 2 |
| 26 | VPY* | 1 |
| 27 | VPV | 1 |
| 28 | YPS | 1 |
| 29 | YPV | 1 |
| 30 | EPI | 1 |

*Triplets common with bovine, equine and ovine species.
**Triplets common with bovine and ovine species.
***In addition the following groups of triplets were included: IPN, LPQ, PIL, VPY, and VPS.

Presented in Table 8 is the list of triplets that represents subgroup that is coded in Human DNA. The 35 different triplets can form 1.033314797×10⁴⁰ combinations. These cover a large pool of information needed. In addition, in Beta casein the triplets form more complicated structures listed below:

(i) QPQPLIYPF (SEQ. ID NO.: 46), (ii) EPIPYG (SEQ. ID NO.: 47), (iii) LPLAQP (SEQ. ID NO.: 48), (iv) LPPQPL (SEQ. ID NO.: 49), (v) LPLPLLQPL (SEQ. ID NO.: 50), (vi) LPVPQP (SEQ. ID NO.: 51), (vii) VPYPQQAVP (SEQ. ID NO.: 52), (viii) QPLAPV (SEQ. ID NO.: 53), (ix) VPQPKVLPIPQQ (SEQ. ID NO.: 54), (x) VPQPIP (SEQ. ID NO.: 55), and (xi) PTIPFFDPQ (SEQ. ID NO.: 56).

Presented poly triplets represent apparently programs coded on Human Beta casein. They are capable to deliver to offspring 39.916.800 combinations of programs or 1.99584×10⁵ programs per one type of cell operating in the human body. Table 9 summarizes peptides coded on human beta casein and peptides coded on Bovine beta casein to show that they are structurally different.

TABLE 9
Comparison of complexed peptides present in
human and bovine beta casein.

| Human peptides: | | Bovine peptides: | |
|---|---|---|---|
| 1. | QPQPLIYPE | 1. | VPFPGPIPNSLP |
| | (SEQ. ID NO.: 57) | | (SEQ. ID NO.: 58) |
| 2. | LPLAQP | 2. | APKPLTQTP |
| | (SEQ. ID NO.: 54) | | (SEQ. ID NO.: 59) |
| 3. | LPVPQP | 3. | VVPPFLQPE |
| | (SEQ. ID NO.: 51) | | (SEQ. ID NO.: 60) |
| 4. | PTIPFFDPQ | 4. | MPFPKYPVEPFT |
| | (SEQ. ID NO.: 56) | | (SEQ. ID NO.: 61) |
| 5. | LPLPLLQPL | 5. | LPLPLL |
| | (SEQ. ID NO.: 50) | | (SEQ. ID NO.: 62) |
| 6. | VPQPKVLPIPQQ | 6. | QPHQPLPPT |
| | (SEQ. ID NO.: 54) | | (SEQ. ID NO.: 63) |
| 7. | VPYPQQAVP | 7. | LPVPQK |
| | (SEQ. ID NO.: 52) | | (SEQ. ID NO.: 64) |
| 8. | QPLAPV | 8. | NPYPQR |
| | (SEQ. ID NO.: 53) | | (SEQ. ID NO.: 65) |
| | | 9. | GPVKGPFPI |
| | | | (SEQ. ID NO.: 66) |

Material presented in Table 9 is shown in sequence as it was coded in Beta casein. The first in the list are the oldest, since they are located at the beginning of the beta casein amino acid sequences. Information in Table 9 confirms the postulate mentioned earlier that substitution mother PRPC by heterospecies is not advisable. The youngest is apparently polypeptide no 8 in Human and 9 in Bovine. Only 5 peptides have common elements for human and Bovine. Remaining peptides are quite different. The comparisons revealed that in different species, proline rich polypeptide, are constructed in similar fashion but their sequences are not related. This information provides the best evidence that they are carry programs coded but their function is not yet recognized. The other interesting finding worth to mention is that among the triplets are few that cross the species barrier, but they are included in programs coded in larger peptides which are totally different in different species.

The proline rich peptide complex (PRPC) of the present invention represents a language governing all living cells in plant and in animal kingdoms and represents a tool through which accumulated information were formed in the past. Presented triplets and small peptides represent complex that the Stem cell is using to deliver to the host needed information.

There exist two types of signals; activating the processes of transmission of information and terminating transmission. The peptides and triplets described in the present invention carry different functions: protects the positive signals from non specific digestion or interaction with host elements that may become disturbed. The signals may become digested by proteolytic enzymes. The instant invention also describes mechanisms which protects the signals from damage before they reach the target. One of such mechanism may be located in the beta casein first 56 peptides. The sequence is shown below in table 10

TABLE 10

```
Hydrophobicity profile of Human amino acids from beta casein.
1 2 3 4 5 6 7 8 9 10 11 12 13 14 15 16 17
M K V L I L A C L V A L A L A R E
1.9 −3.9 4.2 3.8 4.5 3.8 1.8 2.5 3.8 4.2 1.8 3.8 1.8 3.8 1.8 −3.5 −3.5
18 19 20 21 22 23 24 25 26 27 28 29 30 31 32 33 34 35
T I E S L S S S E E S I T E Y K Q K
0.7 4.5 −3.5 −0.8 3.8 −0.8 −0.8 −0.8 −3.5 3.5 −0.8 4.5 −0.7 −3.5 −1.3 −3.9 −3.5 −3.9
36 37 38 39 40 41 42 43 44 45 46 47 48 49 50 51 52
V E K V K H E D Q Q Q G E D E H Q
4.2 −3.5 −3.9 −4.2 −3.9 −3.2 −3.5 −3.5 −3.5 −3.5 −3.5 −0.4 −3.5 −3.5 −3.5 −3.2 −3.5
  53 54 55 56
  D K I Y
−3.4 −3.9 4.5 −1.3
```

The first seventeen amino acids are identical in all tested species and were formed with +positively charged AA. Then, the sequences start to show deviation among different species. Nevertheless the SSS triplets were found besides human in ovine and bovine species. The EE element was found in human, Equine, Bovine and ovine but located in different places. They may represent sort of information that remain important but distorted. However, the distribution hydrophilic and mixed hydrophobic amino acids strongly suggest other possibility. The hydrophobic region is formed between third and 15 amino acid. It may mean that triplets having hydrophilic profile can be bound based on hydrophilic/hydrophilic interaction. AA 16, 17, 18 represent short region for hydrophobic interaction. The area, where hydrophobic triplets can attached to beta casein are between AA 20-21, 23-35, and from 37-54 AA. The distribution of hydrophilic/hydrophobic region generally corresponded to proportion hydrophilic to mixed triplets. Since the casein beta region of interest is Philogenetically very old, this region between the AA, 3-56 may serve host as storage magazine II for members of CN III and CN IV network. This will support idea that host utilizes one protein to carry members of two network to secure better the triplets protection.

The studies leading to discovery the mechanism protecting signals was already described earlier and is repeated here in Table 11. Amino acids QT, V, and DG may serve as an example of PRP protector present in CN V. The identified peptides were synthesized by BIO-SYNTHESIS Inc. Lewisville Tex. USA. Peptides were purified in to 99% of purity their amino acid sequences were confirmed. The scavengers activity ware tested in Spin trap Electromagnetic resonance and their effect on the amyloid formation was tested on adult Lewis rats exposed to induced hypoxia. The data shows that relatively small modifications of peptide A-1 lead to profound changes in its biological property. If small structural changes in carrier peptides lead to activation their biological activity, similar changes may suppress activity or even terminate the signal. This important property is called feed back signal. Similar type of signals ought to operate within PRPC III and PRPC IV network or are coded in CN V. In Table 12 are listed peptides that may be considered as terminating signals.

TABLE 11
Modification the structure of the peptide that
modulates the biological property of informers.

| | | | Inhibit |
|---|---|---|---|
| | | Scavenger | Amyloid |
| Peptide | Amino Acid Sequence | Activity | Formation |
| A-1 | LQTPQPLLQVMMEPQGD | 11% | 0 |
| | (SEQ. ID NO.: 1) | | |
| A-10 | QPLLQVMMEPQ | 75% | +++++ |
| | (SEQ. ID NO.: 10) | | |
| B-1 | VESYVPLFP | 63% | ++ |
| | (SEQ. ID NO.: 11) | | |
| B-15 | ESYVPLFP | 24% | 0 |
| | (SEQ. ID NO.: 74) | | |

TABLE 12
List of triplets candidates for suppressor activity.

1. DKE, 2. LNF, 3. KYK, 4. LQE, 5. VVM, 6. VVV, 7. AFL, 8. LYQ,
9. EHM, 10. MFV, 11. TDR, 12. DDD, 13. TEE, 14. YQQ, 15. GFG,
16. LQS, 17. GGK, 18. ESQ, 19. GRV, 20. VEE, 21. IGN, 22. FFQ.
23. RMF. 24. MHH. 25. NTE, 26. GD, 27. LQ, and 28. TP.

The triplets listed in Table 12 have the capability to form many combinations protecting triplets and PRP combinations against premature interactions. The large number of possible combination permits to believe they may serve in host as suppressor of informer's network. This finding opens a totally new field for screening of compounds that will control all inflammatory processes in the body as well as control the development neoplastic diseases. This is one possibility. The other possibility is that the triplets, formed in the biological computer memory, permits cell recovery from environmental insults.

PRP in the therapy of Alzheimer's disease (AD): Substitution of synthetic drugs of broad specificity, by the pools of peptides, providing forbearers of knowledge, as described herein heralds the beginning the new era in a treatment of AD. This is based on the preceding discussion explaining the function and biological significance of the communication system which is intimately involved in prevention and treatment of AD.

The inventors hypothesize that, the key element in dementia are defect that leads to the failure to program the new neuronal population that will substitute the lost ones. The model postulates, the presence in the human body five different networks assigned for delivery information. They control functions of the cells, tissues, glands, organs and whole body. In nature, the offspring receive the programs through the communication networks, carrying the ancestor's knowledge, delivered with the mother's colostrums and milk. Defects in programming can occur in newborn neurons, among adults, aging individuals with chronic, progressive, degenerative diseases. Restoration program, for the newborn neuron, provides a pool of information through the communication system, activating cell biological computer. The information for new born neurons ought to be delivered, in similar manner as mother activated the offspring immediately after the birth. There are many reasons the carriers of information fail to rich the newborn neurons, oxidative stresses, lack of the carriers (the PRPC or Triplets), failure of the stem cell to deliver informers, and problem with delivery system can aggravate or cause the problem.

The present inventors have developed, two new complexes carrying informer peptides and natural scavengers. The first, called Bio®-6 was formulated from animal peptides and plant derived scavengers of free radicals. The formula control overproduction of free radicals in cells and neurons damaged by EI and protects damaged cells and neurons from apoptosis.

The second complex, called Bio®-7, was formulated from sixty nine human triplets and peptides-information-carrier. This formula is specially designed for providing new neurons with essential information and for securing its normal function.

Both complexes are capable to provide needed instructions how to repair damages done to the brain tissue and stopped damages induced by free radicals.

According to the present invention the first complex will protect the host from premature depletion the neurons and stimulate the repair process. In AD patients the triplets present in second complex will provide the host information how to repair already existing damages. The complex I has only 24% of peptides having common structures with human, but it is enriched with scavengers of free radicals. During this period of accelerated aging free radical play crucial role in destruction the brain neurons. The Bio-6 combination comes out as superb tool for prevention the AD.

The Bio-7, formula is constructed exclusively with human peptides that are capable to participate in 10⁹⁸ combinations. Such large number of possible combinations provide AD patient, with all information needed by host to repair cellular and metabolic defects that occur during course of disease. The proposed pool of information will surpass the existing current treatments modalities.

AD is a prominent member among disorders characterized by progressive dementia. Besides AD there are other various vascular dementias, like Pick's, Creutzfeldt-Jacobs, Parkinson's, HIV, mad cow disease, and aging. The etiology of AD is not known. However, it is believed, that failure to provide to the neurons, and the information needed for their computers is the basic cause of dementia. Currently available drugs do not treat the root of the problem and tend to miss the etiological target.

The present inventors carried out studies with peptides derived from ovine colostrums. The newly designed Bio-6 complex can be characterized, as strong scavenger complex with triplets having heterospecies characteristics. But delivered through the protecting mechanism described earlier. Laboratory results on AD demonstrated that PRPC (Proline rich peptide complex), isolated from ovine species alters gene expression involved in Amyloid beta (Aβ) precursor protein synthesis. PRPC can protect against oxidative stress and modulate expression of inflammatory chemokines and cytokines. Tau phosphorylation and increased levels of enzymes that proteolitically eliminate amyloid beta (Aβ) has been shown.⁴

The studies, performed with bovine complexes confirmed ovine studies and permit to postulate that Amyloid beta polymerization process is inhibited by PRPC peptides isolated from ovine colostrums. Caspases accelerated by PRPC, cleave amyloid beta as it was shown by A. Rejendran et al. The data presented herein permits the selection of a group of biologically active peptides inhibiting senile plaque formation. The Micro RNA assay analysis, carried with heterospecies PRPC, established where molecular mechanisms are altered during the Alzheimer's disease.⁵ Application the ovine PRPC theoretically can ameliorate clinical situation.⁶ Furthermore PRPC increases the life-spun and neurological responses of mice. Heterospecies proline rich peptides decrease spontaneous and induced mutation in Chinese hamster cells.⁷ Peptides isolated from Colostrums of other species can correct or restore different type of memories in diverse animals.⁸⁻⁹ Studies presented herein suggest that the memory was affected by group of peptides common with peptides of tested animals or the beneficial effect on memory was modulated by scavengers activity present in ovine PRPC.

The networks CN III and CN IV are members of the communication system, prepared from ovine colostrums. They carry only 24% triplets, common with human species. Since these numbers represent summary of two networks of similar size, the cross species reactivity in one network can be estimated on the level of about 12%. Both complexes were used in Bio-6 complexes prepared for research purposes and showing properties described above.

The double blind placebo controlled study, with AD patients and additional study with healthy volunteers, shows that extremely low concentration of complexes have biological activity. The Bio-6 capsules tested on healthy volunteer's shows no signs of toxicity or adverse reactions even after 10 weeks of administration. The oral route of administration normally does not induce the allergy. The human study carried with different group of AD patients for different period of time; 30 weeks, 12, 16, and 28 months and at the dose level of triplets as low as 0.1 mg per patient per every second day reveals changes when compared with the control. Collected variables were analyzed by means of statistical methods like: Student T test, ANOVAs method or one-sided exact Wilcoxon rank sum test.

Regardless of size, type of study and period of administration or statistic analysis the effects of treatment with Bio-6 complexes was similar. The results uniformly showed that oral administration of complexes improved the outcome of AD patients with mild to moderate dementia. In one double blind placebo controlled clinical trial, carried out on 105 AD patients revealed after 15 weeks application of 0.1 mg of PRPC that corresponded to circa 1 mcg per one peptide per person every second day, stabilization of the cognitive function in ADAS-cog on (p=0.02) level. Improvement daily function in IADL was (p=0.02).

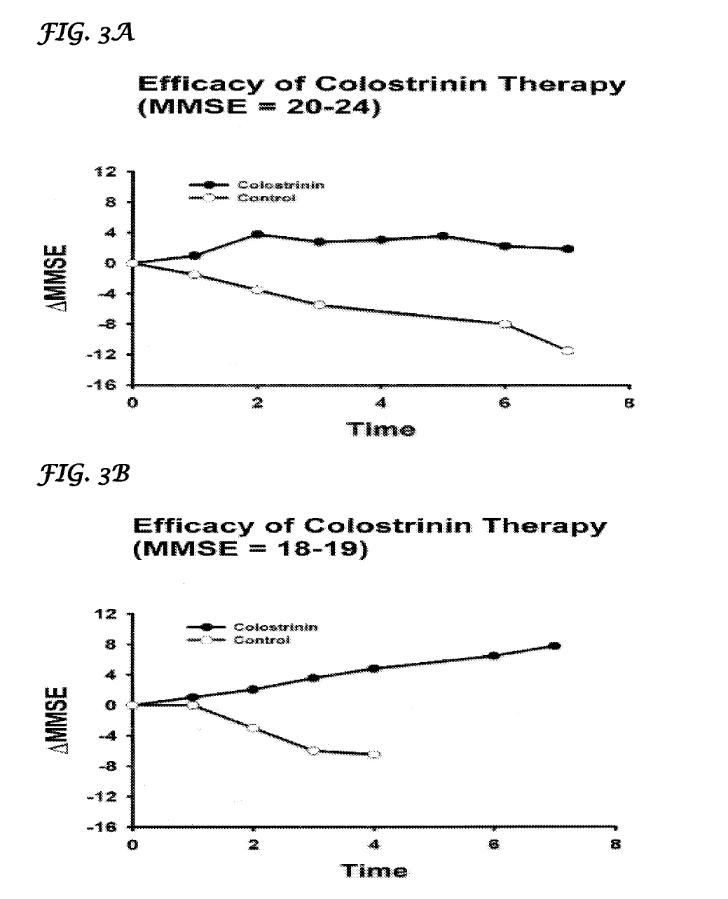

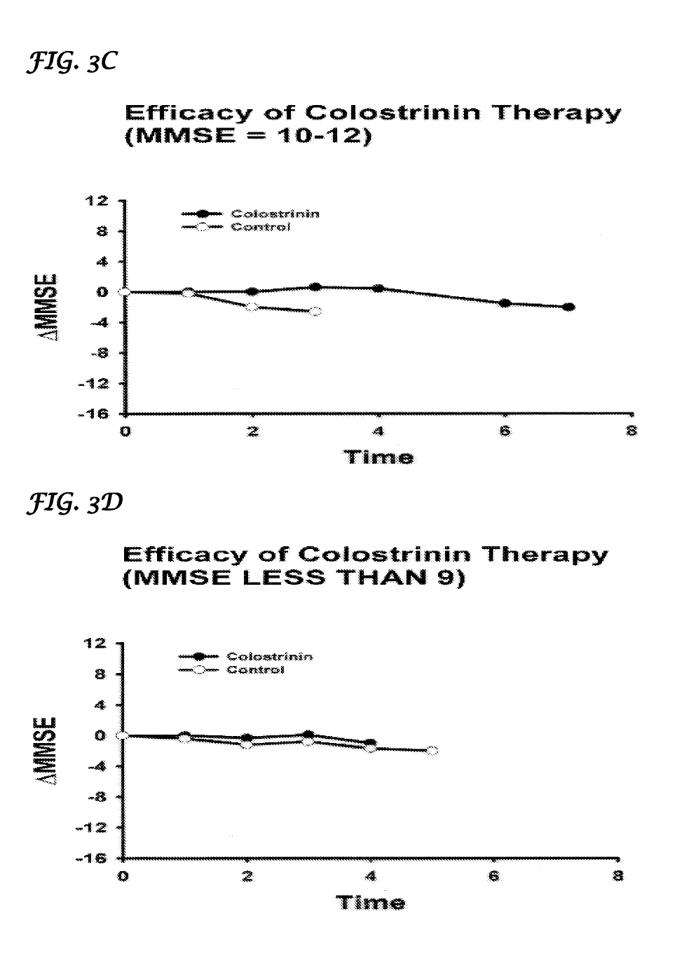

When the whole group of patients were compared, regardless of the state of initial disease, the outcome was also in a favor of the treated (p=0.03). However, when patients, grades were mild on entry, response of ADAS-cog when compared to more advanced cases was (p=0.001).The effect of Bio-6, on the status of the patients with Alzheimer's disease is shown in: FIGS. 3A-3D. The MMSE-Minimal Mental State Examination. Time was measured at bi-monthly intervals.

The Bio-6 of the present invention is derived from natural products used by humans in a daily diet, comprising: Antioxidants (e.g. Vitis viniferous oil, 0.1-50%), Anthocyanes isolated from Aronia berries. 0.001-2%, Hyppohae Ramnoides L. 0.01-10%, Bovine Lactoferrin, 0.1-10% isolated from bovine milk, Flax oil, 0.1-10%, C.L.A, 0.1-10%, Tocoferol acetate, 0.1-10%, Lutein, 1.0-5 mg, and new species of antioxidants and information carriers milk peptides, 0.0001-10%*, inert carriers (P-hydroxybenzoate, 1, 0-20%, Amorphic silica oxide, 1, 0-50% and Fish gelatin to make 0.5 g capsule). The inventors recommend taking one 500 mg capsule every morning.

Four groups of patients at different stage of AD were selected from group of 105 participants assigned to clinical study on efficacy of Bio-6. Each group was divided equally between treated and placebo control. Mini Mental State Examination (MMSE) was the marker for group responses. Data were collected in two month intervals and plotted in FIGS. 3A-3D. Members of the group 1 and 2 treated with colostrinin clearly benefited from using Bio-6. Two remaining groups showed only stabilization effect. Presented results were superior when compared with effect of Dopenesil. Presented data shows effect on early phase of the AD. The results point to the use the Bio-6 complexes for prophylaxis of the AD. These properties were documented in three successive clinical trials. The trend was seen among patient that received Bio-6 for 8, 12, 16, and 28 months of treatment. However, this effect might be interpreted as result of scavengers neutralizing free radicals. For that, this combination was selected for prophylactic purposes, even 24% triplets shared common pattern with human. Furthermore, tested combination shows lack of toxicity (five years of uninterrupted use, data not shown) so the extended use can be recommended without fear of induction of an adverse reaction.

Preparation Bio-7 from the human peptides as restoration therapy for AD patients: The present inventors constructed a complex made with entirely human PRPC that will cover a broad range of information. The list of triplets and short peptides of human origin form new anti AD complex called Bio-7.

List of peptides selected for complex formation:

I. Group 1—Peptides from “PRTB” protein: YPT QPT YPV QPP (SEQ. ID NO.: 67), NPV YPQ (SEQ. ID NO.: 68), LPQ APP (SEQ. ID NO.: 69), YPV QPI YPP (SEQ. ID NO.: 70), and PPP PPG CPP (SEQ. ID NO.: 71).

II. Group 2—Triplets from the “PRTB” protein: APP, CPP, FPG, GPI, HPG, LPM, NPV, PPG, PPP, RPS, QPT, VPT 2X, YPV, and YPQ.

III. Group 3—Common triplets present in different species: FPE, IPN, LPL, LPV, LPQ, MPI, PLL, PKY, PFT, PIL, PQR, QPL 3X, VPQ, VPF, VPP, VPY, and VPV

IV. Group 4—Common triplets present in “PRTB” brain protein and CIII network: LPQ 2X and QPP 2X.

V. Group 5—Triplets derived from CN III network: APV, APQ, DPQ, LPP, LPI, MPV, NPT 3X, PTI, PQQ, PIP, PKV, QPA, VPV, DPK, YPV, PYG, and VPS 2X.

VI. Group 6—Complexes found in CN III group: QPQ PLI YPF (SEQ. ID NO.: 46), LPQ, LPL AQP (SEQ. ID NO.: 48), LPV PQP (SEQ. ID NO.: 51), VPK, PTI PFF DPQ (SEQ. ID NO.: 56), PKL, LPL PLL QPL (SEQ. ID NO.: 50), LPP QPL (SEQ. ID NO.: 49), VPQ PKV LPI

PQQ (SEQ. ID NO.: 54), YPY PQQ AVP (SEQ. ID NO.: 72), QPL RPV (SEQ. ID NO.: 73), and NPI

The Bio-7 was made from Bio-6 to which synthetic human triplets and peptides covering CN III group were added. They are copies of elements present in human being and are used for building cells, tissue, glands organs and whole human body. The Bio-7 capsules consist of the combination described for Bio-6 capsules, including: P-hydroxybenzoate, 1, 0-20%, Aortic Amorfig or Silica oxide powder, 1, 0-5.0%, and fish gelatin to make 0.5 g capsules.

I. The group of Hydrophilic triplets:

| | | Hydrophobicity | Times, Amount per day | | |
|---|---|---|---|---|---|
| Group A: | Triplets, | profile. | in nanograms: Range. | | |
| A. 1 | DPK | −3, 5 −1, 6 −3, 9 | 1 | 650, | (1300-350) |
| A. 1 | DPQ | −3, 5 −1, 6 −3, 5 | 1 | 650, | (1300-350) |
| A. 2 | NPT | −3, 5 −1, 6 −0, 7 | 3 | 1950 | (3000-1000) |
| A. 2 | QPT | −3, 5 −1, 6 −0. 7 | 1 | 650 | (1000-350) |
| A. 2 | RPS | −4, 5 −1, 6 −0, 8 | 1 | 650 | (1000-350) |
| A. 2 | NPT | −3, 5 −1, 6 −0. 7 | 1 | 650 | (1000-350) |
| A. 3 | HPG | −3, 2 −1, 6 −0, 4 | 1 | 650 | (1000-350) |
| A. 4 | QPP | −3, 5 −1, 6 −1. 6 | 3 | 1950 | (3000-1000) |
| A. 5 | PQP | −1, 6 −3, 5 −1, 6 | 2 | 1300 | (2000-500) |
| A. 5 | PKY | −1, 6 −3, 9 −1, 3 | 1 | 650 | (1000-350) |
| A. 6 | PQQ | −1, 6 −3, 5 −3, 5 | 1 | 650 | (1000-350) |
| A. 6 | PQR | −1, 6 −3, 5 −4, 5 | 1 | 650 | (1000-350) |
| A. 7 | PPP | −1, 6 −1, 6 −1, 6 | 1 | 650 | (1000-350) |
| A. 8 | YPQ | −1, 3 −1, 6 −3, 5 | 2 | 1300 | (2000-500) |
| A. 9 | PPG | −1, 6 −1, 6 −0, 4 | 1 | 650 | (1000-350) |
| A. 9 | PYG | −1, 6 −1, 3 −0, 4 | 1 | 650 | (1000-350) |

II. Group B:

| | | | Times: Amount per | |
|---|---|---|---|---|
| Group B: | Amino acid | | day in nanograms: | |
| Groups: | composition | Hydrophobicity profile | Range: | |
| B. 1 | APP | +1, 8 −1, 6 −1, 6 | 1 | 650 (1000-350) |
| B. 1 | CPP | +2, 5 −1, 6 −1, 6 | 1 | 650 (1000-350) |
| B. 1 | FPG | +2, 8 −1. 6 −1, 6 | 1 | 650 (1000-350) |
| B. 2 | VPP | +4, 2 −1, 6 −1, 6 | 1 | 650 (1000-350) |
| B. 2 | VPY | +4, 2 −1, 6 −1, 3 | 1 | 650 (1000-350) |
| B. 2 | VPS | +4, 2 −1, 6 −0, 8 | 2 | 1300 (2000-500) |
| B. 2 | LPP | +3, 8 −1, 6 −1, 6 | 1 | 650 (1000-350) |
| B. 3 | APV | +1, 8 −1, 6 +4, 2 | 1 | 650 (1000-350) |
| B. 3 | MPV | +1, 9 −1, 6 +4, 2 | 1 | 650 (1000-350) |
| B. 4 | GPI | −0, 4 −1, 6 +4.5 | 1 | 650 (1000-350) |
| B. 5 | YPV | −1, 3 −1, 6 +4, 2 | 2 | 1300 (1000-350) |
| B. 6 | PTI | −1, 6 −0, 7 +4, 5 | 1 | 650 (1000-350) |
| B. 7 | LPM | +3, 8 −1, 6 +1, 9 | 1 | 650 (1000-350) |
| B. 8 | QPA | −3, 5 −1, 6 +1, 8 | 1 | 650 (1000-350) |
| B. 9 | NPV | −3, 5 −1, 6 +4, 2 | 1 | 650 (1000-350) |
| B. 9 | VPT | −3, 5 −1, 6 +4, 2 | 2 | 1300 (2000-500) |
| B. 9 | NPI | −3, 5 −1, 6 +4, 5 | 1 | 650 (1000-350) |
| B. 10 | APQ | +1, 8 −1. 6 −3, 5 | 1 | 650 (1000-350) |
| B. 10 | FPE | +2, 8 −1, 6 −3, 5 | 1 | 650 (1000-350) |
| B. 11 | LPQ | +3.8 −1, 6 −3, 5 | 4 | 2600 (4000-1500) |
| B. 11 | VPQ | +4, 2 −1, 6 −3, 5 | 2 | 1300 (2000-500) |
| B. 11 | IPN | +4. 5 −1, 6 −3.5 | 1 | 650 (1000-350) |
| B. 11 | VPK | +4, 2 −1, 6 −3, 9 | 1 | 650 (1000-350) |
| B. 12 | PFT | −1, 6 +2, 8 −0, 7 | 1 | 650 (1000-350) |
| B. 13 | PIP | −1, 6 +4, 5 −1, 6 | 1 | 650 (1000-350) |
| B. 14 | LPL | +3.8 −1, 6 +3, 8 | 1 | 650 (1000-350) |
| B. 14 | LPV | +3.8 −1, 6 +4, 2 | 1 | 650 (1000-350) |
| B, 14 | VPV | +4, 2 −1, 6 +4, 2 | 2 | 1300 (2000-500) |
| B. 14 | VPF | +4, 2 −1, 6 +2, 8 | 1 | 650 (1000-350) |
| B. 14 | LPI | +3, 8 −1, 6 +4, 5 | 1 | 650 |
| B. 15 | PLL | −1, 6 +3, 8 +3, 8 | 1 | 650 (1000-350) |
| B. 15 | PIL | −1, 6 +4, 5 +3, 8 | 1 | 650 (1000-350) |
| B. 16 | QPL | −3, 5 −1, 6 +3, 5 | 3 | 1950 (3000-1000) |
| B. 17 | QPA | −3, 5 −1, 6 +1, 8 | 1 | 650 (1000-3500 |
| B. 18 | PKV | −1, 6 −3, 9 +4, 2 | 1 | 650 (1000-350) |
| B. 18 | PKL | −1, 6 −3, 9 +3.8 | 1 | 650 (1000-350) |
| B. 19 | MPI | +1, 9 −1, 6 +4, 5 | 1 | 650 (1000-350) |
| B. 19 | APV | +1, 8 −1, 6 +4, 2 | 1 | 650 (1000-350) |
| B. 20 | PKA | −1, 6 −3, 5 +1, 8 | 1 | 650 (1000-350) |

III.

| List of small peptides*** | | | | | | | | | | | | | Index | Doses/day Range |
|---|---|---|---|---|---|---|---|---|---|---|---|---|---|---|
| 1. | Y | P | T | - Q | P | T | Y | P | V - | Q | P | P | | |
| | −1, 3 | −1, 6 | −07 | −3, 5 | −1, 6 | −07 | −1, 3 | −1, 6 | +4, 2 | −3, 5 | −1, 6 | −1, 6 | −14, 8 | 650 (1000-350) |
| 2. | N | P | V | Y | P | Q | | | | | | | | |
| | −3, 5 | −1, 6 | +4, 2 | −1, 3 | −1, 6 | −3.5 | | | | | | | −7, 3 | 650 (1000-350) |
| 3. | L | P | Q | A | P | P | | | | | | | | |
| | +3, 8 | −1, 6 | −3, 5 | +1.8 | −1.6 | −1, 6 | | | | | | | −10, 0 | 650 (1000-350) |
| 4. | Y | P | V | Q | P | I | Y | P | P | | | | | |
| | −1, 3 | −1, 6 | +4, 2 | −3, 5 | −1.6 | +4, 2 | +1, 3 | −1, 6 | −1, 6 | | | | −4, 1 | 650 (1000-350) |
| 5. | P | P | P | P | P | G | C | P | P | | | | | |
| | −1, 6 | −1, 6 | −1, 6 | −1, 6 | −1, 6 | −0, 4 | +2, 5 | −1, 6 | −1, 6 | | | | −9, 1 | 650 (1000-350) |
| 6. | Q | P | Q- | P | L | I | Y | P | F | | | | | |
| | −3, 5 | −1, 6 | −3, 5 | −1, 6 | +3, 8 | +4, 5 | −1, 3 | −1, 6 | +2, 8 | | | | −2, 0 | 650 (1000-350) |
| 7. | L | P | L- | P | Q | P | | | | | | | | |
| | +3, 8 | −1, 6 | +3, 8 | −1, 6 | −3, 5 | −1, 6 | | | | | | | −0.7 | 650 (1000-350) |
| 8. | V | P | Q- | P | K | V | -L | P | I- | P | Q | Q | | |
| | +4, 2 | −1, 6 | −3, 5 | −1, 6 | −3, 9 | +4, 2 | +3.8 | −1, 6 | +4, 5 | −1, 6 | −3, 5 | −3, 5 | −2, 9 | 650 (1000-350) |
| 9. | Y | P | Y | P | Q | Q | A | V | P | | | | | |
| | −1, 3 | −1, 6 | −1, 3 | −1.6 | −3, 5 | −3, 5 | +1, 8 | +4, 2 | −1, 6 | | | | −8, 4 | 650 (1000-350) |
| 10. | Q | P | L | R | P | V | | | | | | | | |
| | −3, 5 | −1, 6 | +3, 8 | −4, 5 | −1, 6 | +4, 2 | | | | | | | −3, 2 | 650 (1000-350) |

IV. Amino acids from beta casein. **** The natural carriers for triplets

### Group A.

| | | | | | | | | | | | | | | | | |
|---|---|---|---|---|---|---|---|---|---|---|---|---|---|---|---|---|
| 2 | 3 | 4 | 5 | 6 | 7 | 8 | 9 | 10 | 11 | 12 | 13 | 14 | 15 | 16 | 17 | 18 |
| K | V | L | I | L | A | C | L | V | A | L | A | L | A | R | E | T |
| −3.9 | 4.2 | 3.8 | 4.5 | 3.8 | 1.8 | 2.5 | 3.8 | 4.2 | 1.8 | 3.8 | 1.8 | 3.8 | 1.8 | −3.5 | −3.5 | −0.7 |

### Group B.

| | | | | | | | | | |
|---|---|---|---|---|---|---|---|---|---|
| 19 | 20 | 21 | 22 | 23 | 24 | 25 | 26 | 27 | 28 |
| I | E | S | L | S | S | S | E | E | S |
| 4.5 | −3.5 | −0.8 | 3.8 | −0.8 | −0.8 | −0.8 | −3.5 | −3.5 | −0.8 |

### Group C.

| | | | | | | |
|---|---|---|---|---|---|---|
| 29 | 30 | 31 | 32 | 33 | 34 | 35 |
| I | T | E | Y | K | Q | K |
| 4.5 | −0.7 | −3.5 | −1.3 | −3.9 | −3.5 | .−3.9 |

### Group D.

| | | | | | | | | | | | | | | | | | | |
|---|---|---|---|---|---|---|---|---|---|---|---|---|---|---|---|---|---|---|
| 36 | 37 | 38 | 39 | 40 | 41 | 42 | 43 | 44 | 45 | 46 | 47 | 48 | 49 | 50 | 51 | 52 | 53 | 54 |
| V | E | K | V | K | H | E | D | Q | Q | Q | G | E | D | E | H | Q | D | K |
| +4.2 | −3.5 | −3.5 | +4.2 | −3.9 | −3.2 | −3.5 | −3.5 | −3.5 | −3.5 | −3.5 | −0.4 | −3.5 | −3.5 | −3.5 | −3.2 | −3.5 | −3.4 | −3.9 |

The delivery of triplets needs to be done through the natural route, i.e., the oral. The triplets ought to be placed on the oral mucosa in their active form free of protecting elements in order to be attached to carrier protein.¹⁰ After the oral administration the peptides and triplets need to be adsorbed onto the surface of large protein like, i.e., gamma globulin or beta casein fragments, this adsorption provides protection from premature cleavage by circulating proteases or premature interaction with undesirable elements unless they are given orally with carrier peptides. Proteins that carry the triplets to the cells are also protected from non specific proteases by attached triplets.

The activation programming of the newborn cells in the offspring occurs during breastfeeding. The programming of new neurons, in an adult organism, the host to restore the loss of old neurons population that died due to variety of reasons, like the age or battle with environmental insults. The adsorbed to macromolecules and triplets circulate in the body fluids until they reach a body site that requires them. Changes in the pH, salt concentration, elevated temperature or concentration of free radicals are apparently markers of the desorption process. In conjunction with the carriers, they are delivered, in required concentrations and compositions into the cells and initiate the process of programming the biological computer. The hosts own repair mechanisms will utilize triplet messages and restore the programming process.

As described earlier, the hypothesis was tested earlier, using ovine triplets. The results confirmed the hypotheses presented above. The present invention opens possibilities, to correct other defects of CNS like, errors in logical thinking, where emotions dominate over rational behavior and thought. There are several other candidates for treatment with triplets like: paranoia, depression, and bipolar disorder. The learning mechanisms of the present invention can be considered as a general phenomenon applicable not necessary to group of people with dementia but also to other chronic progressive degenerative diseases like learning disorder, autism or others.

To enrich the goal of modulation AD, 19 members of the human peptides forming human specific subgroup in CN III, was selected. They are capable to participate in 1.216451004×10¹⁷ combinations. Since the pool look little to small, other group of triplets who exhibit cross species property was included. Addition increased the pool by 17 triplets raising the size of the pool 36 members. Such combined group can participate in 3.719933268×10⁴¹ combinations.

Further enrichment the triplet pool was accomplished by including the complex of peptides coded in brain protein called “PRTB”. This complex extended the poll by 14 new members increasing the number of triplets to 55. Such pool of informer's can participate in 1.269640335×10⁷³ reactions. Addition to this group, the peptides coded in human casein increased the group to 69 that permit to participate in 1.711224524×10⁹⁸ possible combinations. The enormous numbers presented above is essential for the formation of an efficacious complex. The human body is formulated by circa 200 different cell types and the organism is formed from 10¹⁷ cells. Sixty nine different triplets represent a small number of triplets per different cell type (0.35 peptide/cell). The complex needs to securely deliver the needed information to every type of cell in the body. That means; one cell in the body, can receive an average 5.76 peptides. This calculation does not precluded formation large number of combination mentioned earlier.

The numbers are not large. Furthermore, AD exhibits so many different defects that it is necessary to provide the host with maximally large pool of available information in order to successfully repair the damage. The suggested dosage of 1 μg per daily dose per 10¹⁷ cells is not used for any type of treatment, modulation or prophylaxis. 1 μg of triplets will deliver 6.022×10¹⁸ molecules to cell of the host. That means 16 molecules per single cell. If to this number we will add molecules from triplets from complexes of supporting triplets the number of molecules may increase by factors of 3, 4, 6, 8 or 10. This will increase the signaling power per 1 cell considerably. To this equation it is necessary to add amplification property of triplets (like in case of interferon).¹¹ The present inventors suggest the application of a formula carrying 100% human peptides will be more efficient and much safer than animal complexes, because it covers larger numbers of information that better restores the metabolism and physiological functions in a host suffering from AD.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 6,500,798: Use of colostrinin, constituent peptides thereof, and analogs thereof, as oxidative stress regulators.
U.S. Pat. No. 7,119,064: Use of colostrinin, constituent peptides thereof, and analogs thereof as modulators of intracellular signaling molecules.
1. P. Popik et al. Cognitive effects of Colostral-Val nonapeptide in aged rats. Behavioral Brain Research 118, 201 2001.
2. Agata Sokolowska et al. Colostrums from different mammal species—A rich source of colostrinin; International Diary J. 2007.
3. Marian Kruzel; Immunomodulation by Nutrition: A New Challenge for Milk-Derived Proteins and Peptides. Anti-Aging Medical Therapeutics: 5 2005.
4. I. Boldogh et al. Colostrinin Increases the life spun and Neurological performance of mice. New trends in Alzheimer's and Parkinson Disorders. Salzburg, Austria, Mar. 14-18, 2007.
5. I. Boldogh, et al. Modulation of 4-HNE-mediated signaling by proline-rich peptides from ovine Colostrums. J. Mol. Neuroscience; 20. 125. 2003.
6. I. Boldogh, M. Kruzel. Colostrinin: An Oxidative Stress Modulator for Prevention and treatment of Age-related Diseases. J. Alzheimer's Disease 12, 1-19. 2007
7. Attila Bacsi et al. Colostrinin decreases spontaneous and induced mutation frequencies at the hprt locus in Chinese hamster V79 cells. J. Exp. Therapeutics and Oncology. 5. 249.2006.
8. P. Popik. et al. Colostrinin, a polypeptide isolated from the early milk Facilitates Learning and Memory in Rats. Pharmacology, Biochemistry and Behavior; 64, 183, 1999.
9. MG. Stewart, D. Banks Enhancement of long-term memory retention by Colostrinin in one-day-old chicks trained on a weak passive avoidance learning paradigm. Neurobiology of Learning and Memory; 86. 66. 2006.
10. J. A. G., W. R. Fleishmann Jr. Oral application of cytokines Biotherapy 8: 205 1996.
11. J. A. G. Oral Cavity Gate of Entry in to the Communication Network. Biotherapy. 11. 39. 1998

What is claimed is:

1. A nutritional composition comprising:
one or more antioxidants derived from one or more animal or plant peptides;
one or more milk peptides;
one or more optional naturally occurring constituents, wherein the naturally occurring constituents comprise amino acids, vitamins, oils, fatty acids, pigments, medicinal herbs and fruits, caretonoids, and combinations thereof; and
one or more optional inert agents or additives, wherein the inert agents or additives comprise excipients, preservatives, bulking agents, gelatin, coloring agents, and combinations thereof.

2. The composition of claim 1, wherein the composition is used to prevent Alzheimer's disease, vascular dementias, Pick's disease, Creutzfeldt-Jacobs disease, Parkinson's disease, HIV, mad cow disease, and premature aging.

3. The composition of claim 1, wherein the composition prevents the depletion of one or more neurons and stimulates repair of one or more damaged neurons.

4. The composition of claim 1, wherein the peptide is bovine lactoferrin.

5. The composition of claim 1, wherein the one or more milk peptides comprise amino acid triplets, amino acid complexes or both.

6. The composition of claim 5, wherein the amino acid triplets, the amino acid complexes or both comprise amino acid proline as one or all of the triplet or the complex.

7. The composition of claim 5, wherein the triplets, amino acid complexes or both are comprised in one or more cellular signaling or communication mechanisms.

8. The composition of claim 5, wherein the amino acid triplets comprise APV, PIP, APQ, PKV, DPQ, PYG, LPP, QPA, LPI, VPV, MPV, DPK, NPT 3X, VPS 2X, PQP 2X, YPV, PTI, PQQ, and combinations thereof.

9. The composition of claim 5, wherein the amino acid complexes comprise QPQ PLI YPF (SEQ. ID NO.: 46), VPQ PKV LPI PQQ (SEQ. ID NO.: 54), LPQ, YPY PQQ AVP (SEQ. ID NO.: 72), LPL AQP (SEQ. ID NO.: 54), QPL RPV (SEQ. ID NO.: 73), LPV PQP (SEQ. ID NO.: 51), NPI, VPK, PTI PFF DPQ (SEQ. ID NO.: 56), PKL, LPL PLL QPL (SEQ. ID NO.: 50), LPP QPL (SEQ. ID NO.: 49), and combinations thereof.

10. The composition of claim 5, wherein the triplets, the amino acid complexes or both may be derived from β-casein.

11. The composition of claim 10, wherein the triplets, the amino acid complexes or both comprise amino acid proline as one or all of the triplet or the complex.

12. The composition of claim 10, wherein the amino acid triplets derived from β-casein comprise at least 3 or more amino acid triplets selected from APV, AQP, AVP, DPQ, LPL, LPV, LPP, LPI, MPV, NPT, and combinations thereof.

13. The composition of claim 10, wherein the amino acid complexes derived from β-casein comprise QPQPLIYPF (SEQ ID NO.: 46), VPYPQQAVP (SEQ ID NO.: 52), EPIPYG (SEQ ID NO.: 47), LPLAQP (SEQ ID NO.: 48), QPLAPV (SEQ ID NO.: 53), VPQPKVLPIPQQ (SEQ ID NO.: 54), LPPQPL (SEQ ID NO.: 49), VPQPIP (SEQ ID NO.: 55), LPLPLLQPL (SEQ ID NO.: 50), PTIPFFDPQ (SEQ ID NO.: 56), LPVPQP (SEQ ID NO.: 51), and combinations thereof.

14. The composition of claim 1, wherein the composition is a capsule, a tablet, a powder, a drink, a baby formula, or any suitable orally consumable form.

15. A composition for preventing Alzheimer's disease comprising:

| | |
|---|---|
| Vitis viniferous oil | 0.1-50%; |
| Anthocyanes isolated from Aronia berries | 0.001-2%; |
| Hyppohae Ramnoides L. | 0.01-10%; |
| Bovine Lactoferrin | 0.1-10%; wherein the |
| | bovine lactoferrin is |
| | isolated from bovine |
| | milk; |
| Flax oil | 0.1-10%; |
| Conjugated Linoleic Acid (C.L.A) | 0.1-10%; |
| Tocoferol acetate | 0.1-10%, |
| Lutein | 1.0-5 mg; |
| Antioxidants and milk peptides | 0.0001-10%; |
| P-hydroxybenzoate, 1 | 0-20%; |
| Amorphic silica oxide, 1 | 0-50%; and |
| Fish gelatin to make a 0.5 g capsule. | |

16. The composition of claim 15, wherein the composition is used to prevent or mitigate the symptoms of vascular dementias, Pick's disease, Creutzfeldt-Jacobs disease, Parkinson's disease, HIV, mad cow disease, and aging.

17. The composition of claim 15, wherein the one or more milk peptides comprise amino acid triplets, amino acid complexes or both.

18. The composition of claim 17, wherein the triplets, the amino acid complexes or both comprise amino acid proline as one or all of the triplet or the complex.

19. The composition of claim 17, wherein the triplets, amino acid complexes or both are comprised in one or more cellular signaling or communication mechanisms.

20. The composition of claim 17, wherein the triplets comprise APV, PIP, APQ, PKV, DPQ, PYG, LPP, QPA, LPI, VPV, MPV, DPK, NPT 3X, VPS 2X, PQP 2X, YPV, PTI, PQQ, and combinations thereof.

21. The composition of claim 17, wherein the amino acid complexes comprise QPQ PLI YPF (SEQ. ID NO.: 46), VPQ PKV LPI PQQ (SEQ. ID NO.: 54), LPQ, YPY PQQ AVP (SEQ. ID NO.: 72), LPL AQP (SEQ. ID NO.: 54), QPL RPV (SEQ. ID NO.: 73), LPV PQP (SEQ. ID NO.: 51), NPI, VPK, PTI PFF DPQ (SEQ. ID NO.: 56), PKL, LPL PLL QPL (SEQ. ID NO.: 50), LPP QPL (SEQ. ID NO.: 49), and combinations thereof.

22. The composition of claim 17, wherein the triplets, the amino acid complexes or both may be derived from β-casein.

23. The composition of claim 22, wherein the triplets, the amino acid complexes or both comprise amino acid proline as one or all of the triplet or the complex.

24. The composition of claim 22, wherein the triplets derived from β-casein comprise at least 3 or more amino acid triplets selected from APV, AQP, AVP, DPQ, LPL, LPV, LPP, LPI, MPV, NPT, and combinations thereof.

25. The composition of claim 22, wherein the amino acid complexes derived from β-casein comprise QPQPLIYPF (SEQ ID NO.: 46), VPYPQQAVP (SEQ ID NO.: 52), EPIPYG (SEQ ID NO.: 47), LPLAQP (SEQ ID NO.: 48), QPLAPV (SEQ ID NO.: 53), VPQPKVLPIPQQ (SEQ ID NO.: 54), LPPQPL (SEQ ID NO.: 49), VPQPIP (SEQ ID NO.: 55), LPLPLLQPL (SEQ ID NO.: 50), PTIPFFDPQ (SEQ ID NO.: 56), LPVPQP (SEQ ID NO.: 51), and combinations thereof.

26. The composition of claim 15, wherein the composition is administered once daily.

27. A method of preventing Alzheimer's Disease (AD) in a human subject comprising the steps of:
identifying the human subject in need of prevention of AD; and
administering a composition once daily to the human subject for the prevention of AD, wherein the composition comprises one or more antioxidants, one or more free radical scavengers derived from animal or plant peptides, one or more milk peptides, and one or more optional amino acids, vitamins, oils, fatty acids, pigments, medicinal herbs and fruits, caretonoids, inert agents, additives, and combinations thereof.

28. The method of claim 27, wherein the one or more milk peptides comprise amino acid triplets, amino acid complexes or both.

29. The method of claim 28, wherein the triplets, the amino acid complexes or both comprise amino acid proline as one or all of the triplet or the complex.

30. The method of claim 28, wherein the triplets, amino acid complexes or both are comprised in one or more cellular signaling or communication mechanisms.

31. The method of claim 28, wherein the amino acid triplets, the amino acid complexes or both may be derived from β-casein.

32. A nutritional composition comprising:
one or more antioxidants derived from one or more animal or plant peptides;
one or more synthetic or natural milk peptides, wherein the peptides comprise one or more amino acid triplets, one or more amino acid complexes or both;
one or more optional naturally occurring constituents, wherein the naturally occurring constituents comprise amino acids, vitamins, oils, fatty acids, pigments, medicinal herbs and fruits, caretonoids, and combinations thereof; and
one or more optional inert agents or additives, wherein the inert agents or additives comprise excipients, preservatives, bulking agents, gelatin, coloring agents, and combinations thereof.

33. The composition of claim 32, wherein the composition is used to mitigate the symptoms of Alzheimer's disease (AD), vascular dementias, Pick's disease, Creutzfeldt-Jacobs disease, Parkinson's disease, HIV, mad cow disease, and aging.

34. The composition of claim 32 wherein the composition repairs of one or more damaged neurons and prevents damage induced by one or more free radicals.

35. The composition of claim 32, wherein the peptide is bovine lactoferrin.

36. The composition of claim 32, wherein the triplets, the amino acid complexes or both comprise amino acid proline as one or all of the amino acids triplet or the complex.

37. The composition of claim 32, wherein the triplets, amino acid complexes or both are comprised in one or more cellular signaling or communication mechanisms.

38. The composition of claim 32, wherein the amino acid triplets comprise, peptides from the complex, APP, CPP, FPG, GPI, HPG, LPM, NPV, PPG, PPP, RPS, QPT, VPT 2X, YPV, YPQ, FPE, IPN, LPL, LPV, LPQ, MPI, PLL, PKY, PFT, PIL, PQR, QPL 3X, VPQ, VPF, VPP, VPY, VPV, APV, APQ, DPQ, LPP, LPI, MPV, NPT 3X, PTI, PQQ, PIP, PKV, QPA, VPV, DPK, YPV, PYG, VPS 2X, LPQ 2X, QPP 2X, PKL, NPI, and combinations thereof.

39. The composition of claim 32, wherein the amino acid complexes comprise YPT QPT YPV QPP (SEQ. ID NO.: 67), NPV YPQ (SEQ. ID NO.: 68), LPQ APP (SEQ. ID NO.: 69), YPV QPI YPP (SEQ. ID NO.: 70), PPP PPG CPP (SEQ. ID NO.: 71), LPL PLL QPL (SEQ. ID NO.: 50), LPP QPL 3X (SEQ. ID NO.: 49), VPQ PKV LPI PQQ (SEQ. ID NO.: 54), YPY PQQ AVP (SEQ. ID NO.: 72), QPL RPV (SEQ. ID NO.: 73), QPQ PLI YPF (SEQ. ID NO.: 46), LPQ, LPL AQP (SEQ. ID NO.: 48), LPV PQP (SEQ. ID NO.: 51), VPK, PTI PFF DPQ (SEQ. ID NO.: 56), and combinations thereof.

40. The composition of claim 32, wherein the composition is a capsule, a tablet, a powder, a drink, a baby formula, or any suitable orally consumable form.

41. A composition for mitigating the symptoms of Alzheimer's disease (AD) comprising:

| | |
|---|---|
| Vitis viniferous oil | 0.1-50%; |
| Anthocyanes isolated from Aronia berries | 0.001-2%; |
| Hyppohae Ramnoides L. | 0.01-10%; |
| Bovine Lactoferrin | 0.1-10%; wherein the |
| | bovine lactoferrin is |
| | isolated from bovine |
| | milk; |
| Flax oil | 0.1-10%; |
| Conjugated Linoleic Acid (C.L.A) | 0.1-10%; |
| Tocoferol acetate | 0.1-10%, |
| Lutein | 1.0-5 mg; |

Synthetic or milk peptides in an amount sufficient to prevent or mitigate the symptoms of AD, wherein the peptides comprise one or more amino acid triplets, one or more amino acid complexes or both;

| | |
|---|---|
| P-hydroxybenzoate, 1 | 0-20%; |
| Amorphic silica oxide, 1 | 0-50%; and |
| Fish gelatin to make a 0.5 g capsule. | |

42. The composition of claim 41, wherein the composition is used to prevent, treat or mitigate the symptoms of vascular dementias, Pick's disease, Creutzfeldt-Jacobs disease, Parkinson's disease, HIV, mad cow disease, and aging.

43. The composition of claim 41, wherein the triplets, the amino acid complexes or both comprise amino acid proline as one or all of the triplet or the complex.

44. The composition of claim 41, wherein the triplets, amino acid complexes or both are comprised in one or more cellular signaling or communication mechanisms.

45. The composition of claim 41, wherein the triplets comprise, peptides from the PRTB complex, APP, CPP, FPG, GPI, HPG, LPM, NPV, PPG, PPP, RPS, QPT, VPT 2X, YPV, YPQ, FPE, IPN, LPL, LPV, LPQ, MPI, PLL, PKY, PFT, PIL, PQR, QPL 3X, VPQ, VPF, VPP, VPY, VPV, APV, APQ, DPQ, LPP, LPI, MPV, NPT 3X, PTI, PQQ, PIP, PKV, QPA, VPV, DPK, YPV, PYG, VPS 2X, LPQ 2X, QPP 2X, PKL, NPI, and combinations thereof.

46. The composition of claim 41, wherein the amino acid complexes comprise YPT QPT YPV QPP (SEQ. ID NO.: 67), NPV YPQ (SEQ. ID NO.: 68), LPQ APP (SEQ. ID NO.: 69), YPV QPI YPP (SEQ. ID NO.: 70), PPP PPG CPP (SEQ. ID NO.: 71), LPL PLL QPL (SEQ. ID NO.: 50), LPP QPL 3X (SEQ. ID NO.: 49), VPQ PKV LPI PQQ (SEQ. ID NO.: 54), YPY PQQ AVP (SEQ. ID NO.: 72), QPL RPV (SEQ. ID NO.: 73), QPQ PLI YPF (SEQ. ID NO.: 46), LPQ, LPL AQP (SEQ. ID NO.: 48), LPV PQP (SEQ. ID NO.: 51), VPK, PTI PFF DPQ (SEQ. ID NO.: 56), and combinations thereof.

47. The composition of claim 41, wherein the composition is administered once daily.

48. A method for mitigating the symptoms of Alzheimer's Disease (AD) in a human subject comprising the steps of:
identifying the human subject in need of treatment of the AD; and
administering a composition once daily to the human subject in a quantity sufficient for the treatment of the AD, wherein the composition comprises one or more antioxidants, one or more free radical scavengers derived from animal or plant peptides, one or more synthetic or milk peptides comprising amino acids triplets, amino acid complexes or both, and one or more optional amino acids, vitamins, oils, fatty acids, pigments, medicinal herbs and fruits, caretonoids, inert agents, additives, and combinations thereof.

49. The method of claim 48, further comprising the steps of:
monitoring the progression of the mitigation by measuring a cognitive state of the human subject at regular intervals by performing a mini mental state examination (MMSE) and other test scores used for an evaluation of cognitive state of the human subject;
altering the quantity of the composition, administered or stopping the administration of the composition based on a result of the MMSE and other test scores used for the evaluation of the cognitive state of the human subject.

50. The method of claim 48, wherein the triplets, the amino acid complexes or both comprise amino acid proline as one or all of the triplet or the complex.

51. The method of claim 48, wherein the triplets, amino acid complexes or both are comprised in one or more cellular signaling or communication mechanisms.

52. The method of claim 48, wherein the amino acid triplets comprise, peptides from the PRTB complex, APP, CPP, FPG, GPI, HPG, LPM, NPV, PPG, PPP, RPS, QPT, VPT 2X, YPV, YPQ, FPE, IPN, LPL, LPV, LPQ, MPI, PLL, PKY, PFT, PIL, PQR, QPL 3X, VPQ, VPF, VPP, VPY, VPV, APV, APQ, DPQ, LPP, LPI, MPV, NPT 3X, PTI, PQQ, PIP, PKV, QPA, VPV, DPK, YPV, PYG, VPS 2X, LPQ 2X, QPP 2X, PKL, NPI, and combinations thereof.

53. The method of claim 48, wherein the amino acid complexes comprise YPT QPT YPV QPP (SEQ. ID NO.: 67), NPV YPQ (SEQ. ID NO.: 68), LPQ APP (SEQ. ID NO.: 69), YPV QPI YPP (SEQ. ID NO.: 70), PPP PPG CPP (SEQ. ID NO.: 71), LPL PLL QPL (SEQ. ID NO.: 50), LPP QPL 3X (SEQ. ID NO.: 49), VPQ PKV LPI PQQ (SEQ. ID NO.: 54), YPY PQQ AVP (SEQ. ID NO.: 72), QPL RPV (SEQ. ID NO.: 73), QPQ PLI YPF (SEQ. ID NO.: 46), LPQ, LPL AQP (SEQ. ID NO.: 48), LPV PQP (SEQ. ID NO.: 51), VPK, PTI PFF DPQ (SEQ. ID NO.: 56), and combinations thereof.

54. An immunological method for the determination of a titer of one or more triplets, peptides, proline rich peptide complexes (PRPCs) in a colostrum or other biological fluids comprising the steps of:
providing one or more monospecific antibodies against a single amino acid, the triplets, the peptides or the PRPCs, wherein the monospecific antibodies are raised in a rabbit, a horse, a sheep or a goat by injecting the one or more triplets, the PRPCs or both, wherein the triplets, the PRPCs or both may be conjugated with a polymer or non-immunogenic amino acids or peptides;
determining a cross-reactivity of the one or more monospecific antibodies with the amino acids, triplets, the peptides or the PRPCs in an Enzyme Linked Immuno-sorbent Assay (ELISA); and
determining the titer of the amino acids, triplets, the peptides, or the PRPCs based on the extent of cross-reactivity between the monospecific antibodies and the amino acids, the triplets, the peptides or the PRPCs.

55. The method of claim 54, wherein the peptides comprise at least one of LQTPQPLLQVMMEPQGD (SEQ. ID NO.: 1), MPQNFYKLPQM (SEQ. ID NO.: 2), VLEMKFPPPPQETVT (SEQ. ID NO.: 3), LKPFPKLKVEVFPFP (SEQ. ID NO.: 4), SEQP (SEQ. ID NO.: 5), DPPPPQS (SEQ. ID NO.: 6), MIVVRLLQNEVPE (SEQ. ID NO.: 7), SLSQSKVLPV (SEQ. ID NO.: 8), LQTQTPVV (SEQ. ID NO.: 9), QPLLQVMMEPQ (SEQ. ID NO.: 10), VESYVPLFP (SEQ. ID NO.: 11), LPPNVG (SEQ. ID NO.: 12), SEEMP (SEQ. ID NO.: 13), DSQPPV (SEQ. ID NO.: 14), FPPPK (SEQ. ID NO.: 15), DLEMPVLPVEPFPFV (SEQ. ID NO.: 16), LFFFLPVVNVLP (SEQ. ID NO.: 17), MQPPPLP (SEQ. ID NO.: 18), DQPPDVEKPDLQPFQVQS (SEQ. ID NO.: 19), LVYPFTGPIPNSLPQNILP (SEQ. ID NO.: 20), EMPFPKY (SEQ. ID NO.: 21), and 1 Lacto 1.


SEQUENCE LISTING (88 sequences)

SEQ ID NO: 1 - protein, 17 aa, Ovis aries
LQTPQPLLQVMMEPQGD

SEQ ID NO: 2 - protein, 11 aa, Ovis aries
MPQNFYKLPQM

SEQ ID NO: 3 - protein, 15 aa, Ovis aries
VLEMKFPPPPQETVT

SEQ ID NO: 4 - protein, 15 aa, Ovis aries
LKPFPKLKVEVFPFP

SEQ ID NO: 5 - protein, 4 aa, Ovis aries
SEQP

SEQ ID NO: 6 - protein, 7 aa, Ovis aries
DPPPPQS

SEQ ID NO: 7 - protein, 13 aa, Ovis aries
MIVVRLLQNEVPE

SEQ ID NO: 8 - protein, 10 aa, Ovis aries
SLSQSKVLPV

SEQ ID NO: 9 - protein, 8 aa, Ovis aries
LQTQTPVV

SEQ ID NO: 10 - protein, 11 aa, Ovis aries
QPLLQVMMEPQ

SEQ ID NO: 11 - protein, 9 aa, Ovis aries
VESYVPLFP

SEQ ID NO: 12 - protein, 6 aa, Ovis aries
LPPNVG

SEQ ID NO: 13 - protein, 5 aa, Ovis aries
SEEMP

SEQ ID NO: 14 - protein, 6 aa, Ovis aries
DSQPPV

SEQ ID NO: 15 - protein, 5 aa, Ovis aries
FPPPK

SEQ ID NO: 16 - protein, 15 aa, Ovis aries
DLEMPVLPVEPFPFV

SEQ ID NO: 17 - protein, 12 aa, Ovis aries
LFFFLPVVNVLP

SEQ ID NO: 18 - protein, 7 aa, Ovis aries
MQPPPLP

SEQ ID NO: 19 - protein, 18 aa, Ovis aries
DQPPDVEKPDLQPFQVQS

SEQ ID NO: 20 - protein, 19 aa, Ovis aries
LVYPFTGPIPNSLPQNILP

SEQ ID NO: 21 - protein, 7 aa, Ovis aries
EMPFPKY

SEQ ID NO: 22 - protein, 9 aa, Ovis aries
VYPFPGPIN

SEQ ID NO: 23 - protein, 9 aa, Ovis aries
SPSLPQNIL

SEQ ID NO: 24 - protein, 10 aa, Ovis aries
TQTPVVVPPF

SEQ ID NO: 25 - protein, 18 aa, Ovis aries
LQPEIMGVPKVKETMVPK

SEQ ID NO: 26 - protein, 18 aa, Ovis aries
HKEMPFPKYPVEPFTESQ

SEQ ID NO: 27 - protein, 18 aa, Ovis aries
SLTLTDVEKLHLPLPLVQ

SEQ ID NO: 28 - protein, 10 aa, Ovis aries
SWMHQPPQLP

SEQ ID NO: 29 - protein, 14 aa, Ovis aries
MHQPPQPLPPTVMF

SEQ ID NO: 30 - protein, 10 aa, Ovis aries
QPLPPTVMFP

SEQ ID NO: 31 - protein, 5 aa, Ovis aries
PQSVH

SEQ ID NO: 32 - protein, 22 aa, Ovis aries
LSQPKVLPVPQKAVPKPDMPIQ

SEQ ID NO: 33 - protein, 8 aa, Ovis aries
RGPFPILV

SEQ ID NO: 34 - protein, 6 aa, Ovis aries
VPPFLQ

SEQ ID NO: 35 - protein, 4 aa, Ovis aries
PKVK

SEQ ID NO: 36 - protein, 4 aa, Ovis aries
FPPQ

SEQ ID NO: 37 - protein, 6 aa, Ovis aries
PVLGPV

SEQ ID NO: 38 - protein, 13 aa, Ovis aries
AFLLYQEPVLGPV

SEQ ID NO: 39 - protein, 6 aa, Ovis aries
PVEPFT

SEQ ID NO: 40 - protein, 5 aa, Ovis aries
PMFLQ

SEQ ID NO: 41 - protein, 4 aa, Ovis aries
VQPF

SEQ ID NO: 42 - protein, 8 aa, Ovis aries
RGPTPILV

SEQ ID NO: 43 - protein, 6 aa, Ovis aries
PVLGPV

SEQ ID NO: 44 - protein, 9 aa, Homo sapiens
QPQPLIYPF

SEQ ID NO: 45 - protein, 6 aa, Homo sapiens
EPIPYG

SEQ ID NO: 46 - protein, 6 aa, Homo sapiens
LPLAQP

SEQ ID NO: 47 - protein, 6 aa, Homo sapiens
LPPQPL

SEQ ID NO: 48 - protein, 9 aa, Homo sapiens
LPLPLLQPL

SEQ ID NO: 49 - protein, 6 aa, Homo sapiens
LPVPQP

SEQ ID NO: 50 - protein, 9 aa, Homo sapiens
VPYPQQAVP

SEQ ID NO: 51 - protein, 6 aa, Homo sapiens
QPLAPV

SEQ ID NO: 52 - protein, 12 aa, Homo sapiens
VPQPKVLPIPQQ

SEQ ID NO: 53 - protein, 6 aa, Homo sapiens
VPQPIP

SEQ ID NO: 54 - protein, 9 aa, Homo sapiens
PTIPFFDPQ

SEQ ID NO: 55 - protein, 9 aa, Homo sapiens
QPQPLIYPE

SEQ ID NO: 56 - protein, 12 aa, Bos primigenius
VPFPGPIPNSLP

SEQ ID NO: 57 - protein, 9 aa, Bos primigenius
APKPLTQTP

SEQ ID NO: 58 - protein, 9 aa, Bos primigenius
VVPPFLQPE

SEQ ID NO: 59 - protein, 12 aa, Bos primigenius
MPFPKYPVEPFT

SEQ ID NO: 60 - protein, 6 aa, Bos primigenius
LPLPLL

SEQ ID NO: 61 - protein, 9 aa, Bos primigenius
QPHQPLPPT

SEQ ID NO: 62 - protein, 6 aa, Bos primigenius
LPVPQK

SEQ ID NO: 63 - protein, 6 aa, Bos primigenius
NPYPQR

SEQ ID NO: 64 - protein, 9 aa, Bos primigenius
GPVKGPFPI

SEQ ID NO: 65 - protein, 12 aa, Artificial sequence
  Synthetic peptide similar to human proline rich peptide complexes.
YPTQPTYPVQPP

SEQ ID NO: 66 - protein, 6 aa, Artificial sequence
  Synthetic peptide similar to human proline rich peptide complexes.
NPVYPQ

SEQ ID NO: 67 - protein, 6 aa, Artificial sequence
  Synthetic peptide similar to human proline rich peptide complexes.
LPQAPP

SEQ ID NO: 68 - protein, 9 aa, Artificial sequence
  Synthetic peptide similar to human proline rich peptide complexes.
YPVQPIYPP

SEQ ID NO: 69 - protein, 9 aa, Artificial sequence
  Synthetic peptide similar to human proline rich peptide complexes.
PPPPPGCPP

SEQ ID NO: 70 - protein, 9 aa, Artificial sequence
  Synthetic peptide similar to human proline rich peptide complexes.
YPYPQQAVP

SEQ ID NO: 71 - protein, 6 aa, Artificial sequence
  Synthetic peptide similar to human proline rich peptide complexes.
QPLRPV

SEQ ID NO: 72 - protein, 4 aa, Homo sapiens
QPPD

SEQ ID NO: 73 - protein, 4 aa, Homo sapiens
QPPQ

SEQ ID NO: 74 - protein, 4 aa, Homo sapiens
QPPV

SEQ ID NO: 75 - protein, 4 aa, Homo sapiens
QPPF

SEQ ID NO: 76 - protein, 4 aa, Homo sapiens
VPPF

SEQ ID NO: 77 - protein, 5 aa, Homo sapiens
KPFPK

SEQ ID NO: 78 - protein, 5 aa, Homo sapiens
GPIPN

SEQ ID NO: 79 - protein, 5 aa, Homo sapiens
EPFPF

SEQ ID NO: 80 - protein, 5 aa, Homo sapiens
YPFPI

SEQ ID NO: 81 - protein, 5 aa, Homo sapiens
DPPPQ

SEQ ID NO: 82 - protein, 5 aa, Homo sapiens
QPPPQ

SEQ ID NO: 83 - protein, 5 aa, Homo sapiens
FPFPF

SEQ ID NO: 84 - protein, 5 aa, Homo sapiens
VPLFP

SEQ ID NO: 85 - protein, 4 aa, Homo sapiens
GPPF

SEQ ID NO: 86 - protein, 5 aa, Homo sapiens
FPPPQ

SEQ ID NO: 87 - protein, 5 aa, Homo sapiens
TPQPL

SEQ ID NO: 88 - protein, 9 aa, Ovis aries
RPKHPIKHQ